(19) United States
(12) Patent Application Publication
(10) Pub. No.: US 2025/0218534 A1
(43) Pub. Date: Jul. 3, 2025

(54) ARTIFICIAL INTELLIGENCE-BASED EPIGENETICS AT BASE RESOLUTION

(71) Applicant: ILLUMINA, INC., San Diego, CA (US)

(72) Inventors: Kishore Jaganathan, San Francisco, CA (US); Delasa Aghamirzaie, Castro Valley, CA (US); Sofia Kyriazopoulou Panagiotopoulou, Redwood City, CA (US); Kai-How Farh, Hillsborough, CA (US)

(21) Appl. No.: 18/849,916

(22) PCT Filed: Jul. 21, 2023
(86) PCT No.: PCT/US2023/028394

§ 371 Date: Sep. 23, 2024

Related U.S. Application Data
(60) Provisional application No. 63/395,774, filed on Aug. 5, 2022.

(51) Int. Cl. G16B 20/20; G16B 25/10

(52) U.S. Cl. CPC G16B 20/20; G16B 25/10

(57) ABSTRACT

The technology disclosed relates to reliably identifying variants that cause extreme levels of gene expression. Extreme levels of gene expression include under expression and over expression. Then, these variants are used to train artificial intelligence based models for a variety of prediction tasks. One example of the prediction tasks is to produce per-base resolution for chromatin sequences. Another example of the chromatin task is to produce gene expression changes caused by the reliably identified variants.

### CROSS-REFERENCE TO RELATED APPLICATION

This application is related to U.S. Patent Application entitled “ARTIFICIAL INTELLIGENCE-BASED DETECTION OF GENE CONSERVATION AND EXPRESSION PRESERVATION AT BASE RESOLUTION,” filed contemporaneously (Attorney Docket No. ILLM 1036-1/IP-2045-PRV), which is incorporated by reference for all purposes as if fully set forth herein.

### INCORPORATIONS

The following are incorporated by reference for all purposes as if fully set forth herein:

U.S. Patent Application No. 62/903,700, titled “ARTIFICIAL INTELLIGENCE-BASED EPIGENETICS,” filed Sep. 20, 2019 (Attorney Docket No. ILLM 1025-1/IP-1898-PRV);

Sundaram, L. et al. Predicting the clinical impact of human mutation with deep neural networks. Nat. Genet. 50, 1161-1170 (2018);

Jaganathan, K. et al. Predicting splicing from primary sequence with deep learning. Cell 176, 535-548 (2019);

U.S. Patent Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-1/IP-1611-PRV);

U.S. Patent Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV);

U.S. Patent Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV);

U.S. Patent Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV);

U.S. patent application Ser. No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US);

U.S. patent application Ser. No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US);

U.S. patent application Ser. No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US);

U.S. patent application Ser. No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-1/IP-1734-US);

U.S. patent application Ser. No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed on Apr. 15, 2021, (Atty. Docket No. ILLM 1037-2/IP-2051-US);

U.S. Patent Application No. 63/175,495, titled “MULTI-CHANNEL PROTEIN VOXELIZATION TO PREDICT VARIANT PATHOGENICITY USING DEEP CONVOLUTIONAL NEURAL NETWORKS,” filed on Apr. 15, 2021, (Atty. Docket No. ILLM 1047-1/IP-2142-PRV);

U.S. Patent Application No. 63/175,767, titled “EFFICIENT VOXELIZATION FOR DEEP LEARNING,” filed on Apr. 16, 2021, (Atty. Docket No. ILLM 1048-1/IP-2143-PRV); and

U.S. patent application Ser. No. 17/468,411, titled “ARTIFICIAL INTELLIGENCE-BASED ANALYSIS OF PROTEIN THREE-DIMENSIONAL (3D) STRUCTURES,” filed on Sep. 7, 2021, (Atty. Docket No. ILLM 1037-3/IP-2051A-US).

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to artificial intelligence-based epigenetics at base resolution.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genomics, in the broad sense, also referred to as functional genomics, aims to characterize the function of every genomic element of an organism by using genome-scale assays such as genome sequencing, transcriptome profiling, and proteomics. Genomics arose as a data-driven science-it operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions and residues such as transcriptional enhancers and single nucleotide polymorphisms (SNPs).

Genomics data are too large and too complex to be mined solely by visual investigation of pairwise correlations. For example, protein sequences can be classified into families of homologous proteins that descend from an ancestral protein and share a similar structure and function. Analyzing multiple sequence alignments (MSAs) of homologous proteins provides important information about functional and structural constraints. The statistics of MSA columns, representing amino-acid sites, identify functional residues that are conserved during evolution. Correlations of amino acid usage between the MSA columns contain important information about functional sectors and structural contacts.

Instead, analytical tools are required to support the discovery of unanticipated relationships, to derive novel hypotheses and models, and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells, or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, and the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks, and many others.

Logistic regression is a binary classifier, that is, a supervised learning model that predicts a binary target variable. Specifically, logistic regression predicts the probability of the positive class by computing a weighted sum of the input features mapped to the [0,1] interval using the sigmoid function, a type of activation function. The parameters of logistic regression, or other linear classifiers that use different activation functions, are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation followed by sequencing (ChIP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible k-mers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATA1 and TAL1. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATA1 motif and TAL1 motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChIP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequences across distances as long as typical human introns (See Jaganathan, K. et al. Predicting splicing from primary sequence with deep learning. Cell 176, 535-548 (2019)).

Different types of neural networks can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model to each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans. In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to finding potential drivers of complex phenotypes. One example includes predicting the effect of non-coding single-nucleotide variants and short insertions or deletions (indels) indirectly from the difference between two variants in terms of transcription factor binding, chromatin accessibility, or gene expression predictions. Another example includes predicting novel splice site creation from sequence or quantitative effects of genetic variants on splicing.

End-to-end deep learning approaches for variant effect predictions are applied to predict the pathogenicity of missense variants from protein sequence and sequence conservation data (See Sundaram, L. et al. Predicting the clinical impact of human mutation with deep neural networks. Nat. Genet. 50, 1161-1170 (2018), referred to herein as “PrimateAI”). PrimateAI uses deep neural networks trained on variants of known pathogenicity with data augmentation using cross-species information. In particular, PrimateAI uses sequences of wild-type and mutant proteins to compare the difference and decide the pathogenicity of mutations using the trained deep neural networks. Such an approach that utilizes the protein sequences for pathogenicity prediction is promising because it can avoid the circularity problem and overfitting to previous knowledge. However, compared to the adequate number of data to train the deep neural networks effectively, the number of clinical data available in ClinVar is relatively small. To overcome this data scarcity, PrimateAI uses common human variants and variants from primates as benign data while simulated variants based on trinucleotide context were used as unlabeled data.

PrimateAI outperforms prior methods when trained directly upon sequence alignments. PrimateAI learns important protein domains, conserved amino acid positions, and sequence dependencies directly from the training data consisting of about 120,000 human samples. PrimateAI substantially exceeds the performance of other variant pathogenicity prediction tools in differentiating benign and pathogenic de-novo mutations in candidate developmental disorder genes, and in reproducing prior knowledge in ClinVar. These results suggest that PrimateAI is an important step forward for variant classification tools that may lessen the reliance of clinical reporting on prior knowledge.

Central to protein biology is the understanding of how structural elements give rise to observed function. The surfeit of protein structural data enables the development of computational methods to systematically derive rules governing structural-functional relationships. However, the performance of these methods depends critically on the choice of protein structural representation.

Protein sites are microenvironments within a protein structure, distinguished by their structural or functional role. A site can be defined by a three-dimensional (3D) location and a local neighborhood around this location in which the structure or function exists. Central to rational protein engineering is the understanding of how the structural arrangement of amino acids creates functional characteristics within protein sites. Determination of the structural and functional roles of individual amino acids within a protein provides information to help engineer and alter protein functions. Identifying functionally or structurally important amino acids allows focused engineering efforts such as site-directed mutagenesis for altering targeted protein functional properties. Alternatively, this knowledge can help avoid engineering designs that would abolish a desired function.

Since it has been established that structure is far more conserved than sequence, the increase in protein structural data provides an opportunity to systematically study the underlying pattern governing the structural-functional relationships using data-driven approaches. A fundamental aspect of any computational protein analysis is how protein structural information is represented. The performance of machine learning methods often depends more on the choice of data representation than the machine learning algorithm employed. Good representations efficiently capture the most critical information while poor representations create a noisy distribution with no underlying patterns.

The surfeit of protein structures and the recent success of deep learning algorithms provide an opportunity to develop tools for automatically extracting task-specific representations of protein structures.

The computational analysis of genomics studies is challenged by confounding variation that is unrelated to the genetic factors of interest. Identification of variants that cause extreme levels of gene expression, either high or low, is paramount to the diagnosis of the pathogenicity of genetic diseases. However, there are numerous confounding factors that can interfere with the identification of pathogenic variants. Isolating variants by examining rare variants that can be associated with specific pathologies can simplify the problem. Further, removing noise introduced by confounders can increase the signal-to-noise ratio.

Therefore, an opportunity arises to apply artificial intelligence to epigenetics to greatly increase the sensitivity in recovering genetic associations between variable genetic loci and the expression levels of individual genes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1 shows one implementation of a processing pipeline that identifies rare variants that cause extreme levels of gene expression.

FIG. 2 illustrates one implementation of a processing system that generates causality scores for outlier variants that cause extreme levels of gene expression.

FIG. 3 depicts one implementation of a fitted causality model that determines causal relationships between the rare variants and the extreme levels of gene expression while controlling for a plurality of confounders.

FIG. 4 shows examples of causality scores generated by the technology disclosed for a sample of rare variants.

FIG. 5 shows that performance results, measured as a z-score across individuals and genes, improve progressively by successively correcting for the plurality of confounders using the fitted causality model.

FIG. 6 compares the counts of real rare variants and shuffled (or randomly selected) variants that are identified as causing under gene expression and over gene expression for a given p-value from the fitted causality model.

FIG. 7 shows the odds ratios that compare the causality of the real rare variants and the shuffled variants with respect to under gene expression and over gene expression for the given p-value from the fitted causality model.

FIG. 8 shows a first example architecture of the disclosed chromatin model.

FIG. 9 shows a second example architecture of the disclosed chromatin model.

FIG. 10 shows a third example architecture of the disclosed chromatin model.

FIG. 11 shows a fourth example architecture of the disclosed chromatin model.

FIG. 12 shows a fifth example architecture of the disclosed chromatin model.

FIG. 13 illustrates an input generation logic that accesses a sequence database and generates an input base sequence.

FIG. 14 depicts one implementation of base resolution evolutionary conservation prediction by the chromatin model.

FIG. 15 shows an example of a output sequence corresponding to a target base sequence.

FIG. 16 shows one implementation of the disclosed gene expression model.

FIG. 17 shows an example of a reference sequence and an alternate sequence.

FIG. 18 illustrates one implementation of the disclosed variant classification logic.

FIG. 19 illustrates one implementation of the disclosed pathogenicity prediction logic.

FIG. 20 is an example computer system that can be used to implement various aspects of the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

### Example Applications of the Technology Disclosed

Gene expression is the process by which the instructions in DNA are converted into a functional product, such as an RNA molecule or a protein. The technology disclosed identifies rare variants that cause extreme levels of gene expression, which includes both under expression and over expression. The rare variants are identified by association with nearby genes that have the extreme levels of gene expression. In one implementation, the technology disclosed identifies those individuals who have a particular variant in the promoter region of a gene and also have significantly different gene expression for that gene compared to individuals who do not have the particular variant. Based on identifying such individuals, the technology disclosed classifies the particular variant as a gene-expression altering variant. The technology disclosed further uses artificial intelligence to train a plurality of models using the identified rare variants as training data and their phenotype of under expression and over expression as ground truth labels.

In this application, the terms chromatin sequence, chromatin input sequence, and output sequence, are used throughout this application. Chromatin is DNA with bound proteins and/or RNA. By using the term chromatin sequence, we are referring to the DNA sequence of the chromatin. The DNA sequence in a section of chromatin may be protected by protein and RNA and later sequenced as in DNA footprinting. The chromatin sequence may also be chemically modified. For example, DNA sequences often have methyl groups attached to the nucleotides in the sequence and thus are methylated.

As emphasized by the numerous comparisons of various implementations of the chromatin model 124, many implementations share an overlap in architectural components. Each element of the chromatin model 124 has multiple implementations which can be combined in numerous configurations. The multiple permutations which can be implemented for the technology disclosed provides both a broader range of utility, performance efficiency, and performance accuracy. The data transformation applied to the input base sequence in many implementations of the technology disclosed to generate of a plurality of additional sequence formats from the perspective of nucleic acid sequence and the perspective of chromatin structure is an innovative strategy that results in the output of a surfeit of output signals with broad applicability to a wide range of genomics, protein analysis, and pathogenicity research questions. Previous versions of PrimateAI have employed multiple tools for the classification of variant pathogenicity with high performance. This chromatin model 124 introduces another tool in this methodology as well as an additional dimension with the study of epigenetic signals affecting biological replication and transcription processes.

Although there is clear utility for the additional vantage point of chromatin structure to study gene expression to the variety of tools provided by PrimateAI, the true impact provided by the technology disclosed lies in the addition of epigenetic signals to the overall gene expression prediction logic. Both the DNA sequence and histone protein components of chromatin can undergo a plethora of chemical modifications. Enzymes that bind directly to chromatin components and catalyze chemical modifications of the chromatin components can alter chromatin structure, and changes in chromatin structure can also alter the ability of chromatin-interacting enzymes to access their target ligands and function. The structure of chromatin and the enzymes that alter structure directly influence the accessibility of a gene for transcription and expression. DNA variants can cause changes in chromatin structure which subsequently may change epigenetic effects such as transcription factor binding and enzymatic reactions necessary for the proper regulation of gene expression and gene suppression.

Inversely, epigenetic effects on chromatin such as methylation and protein binding events can affect mutation rate, potentially introducing variants that may be silent or pathogenic. The study of evolutionary constraint on a gene and pathogenicity of variants of that gene is significantly more comprehensive and accurate when augmented by epigenetic features as demonstrated in many implementations of the technology disclosed. Overall, the technology disclosed possesses several permutations which are amenable to a range of training and learning strategies to generate several outputs which can be applied to the prediction of gene expression and gene pathogenicity for a target genetic sequence. The chromatin-focused strategy disclosed is useful in the study of inherited and environmental exposure-related disease, the development of drugs, and the influence of epigenetics in the transcription and translation of nucleic acid sequences to proteins.

### Rare Variant Identification

### Processing Pipeline

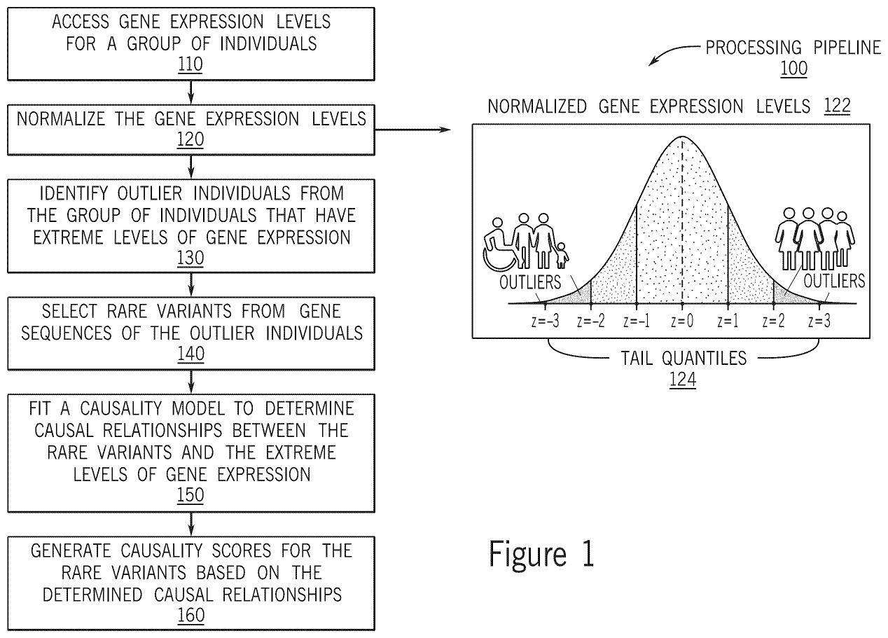

FIG. 1 shows one implementation of a processing pipeline 100 that identifies rare variants that cause extreme levels of gene expression. At action 110, gene expression levels are accessed for a group of individuals. In one implementation, the gene expression levels are accessed from Genotype-Tissue Expression (GTEx).

At action 120, the gene expression levels are normalized, for example, by calculating a mean and a plurality of standard deviations from the mean.

At action 130, those outlier individuals from the group of individuals are identified that have extreme levels of gene expression. The extreme levels of gene expression are determined from tail quantiles 124 of the normalized gene expression levels 122. Examples of the tail quantiles 124 include one or more standard deviations from the mean, both in the positive and the negative directions. For example, the outlier individuals have gene expression levels with z-scores of at least |1.2| from the mean.

At action 140, rare variants from gene sequences of the outlier individuals are selected. The rare variants are selected based on an allele frequency cutoff. For example, the rare variants have a minor allele frequency (MAF) of less than 0.1%.

At action 150, a causality model is fitted to determine causal relationships between the rare variants and the extreme levels of gene expression in the outlier individuals while controlling for a plurality of confounders. In one implementation, the fitted causality model determines the causal relationships by predicting a particular gene expression level of a particular gene in a particular chromosome in dependence upon a variant-driven gene expression level caused by a particular rare variant. In one implementation, the fitted causality model measures a contribution of the variant-driven gene expression level as a variant effect covariate.

Examples of the causality model include a logistic regression model, a linear regression model, an analysis of covariance (ANCOVA) model, and/or a multivariate analysis of covariance (MANCOVA) model. Examples of the plurality of confounders include distal trans-expression quantitative trait loci (eQTLs) effects, local cis-eQTLs effects, genotype-based principal components (gPCs), expression residuals (PEER) effects, environmental effects, population structure and ancestry effects, and gender effects, batch effects, genotyping platform effects, and library construction protocol effects. PEER stands for “probabilistic estimation of expression residuals.” It is a collection of Bayesian approaches to infer hidden determinants and their effects from gene expression profiles using factor analysis methods.

At action 160, causality scores for the rare variants are generated based on the determined causal relationships. A particular causality score of a particular rare variant indicates a likelihood of the particular rare variant causing an extreme level of gene expression in those outlier individuals whose gene sequences contain the particular rare variant. In one implementation, the causality scores are probability values (p-values). In one implementation, the p-values are determined by a Pearson correlation coefficient.

### Processing System

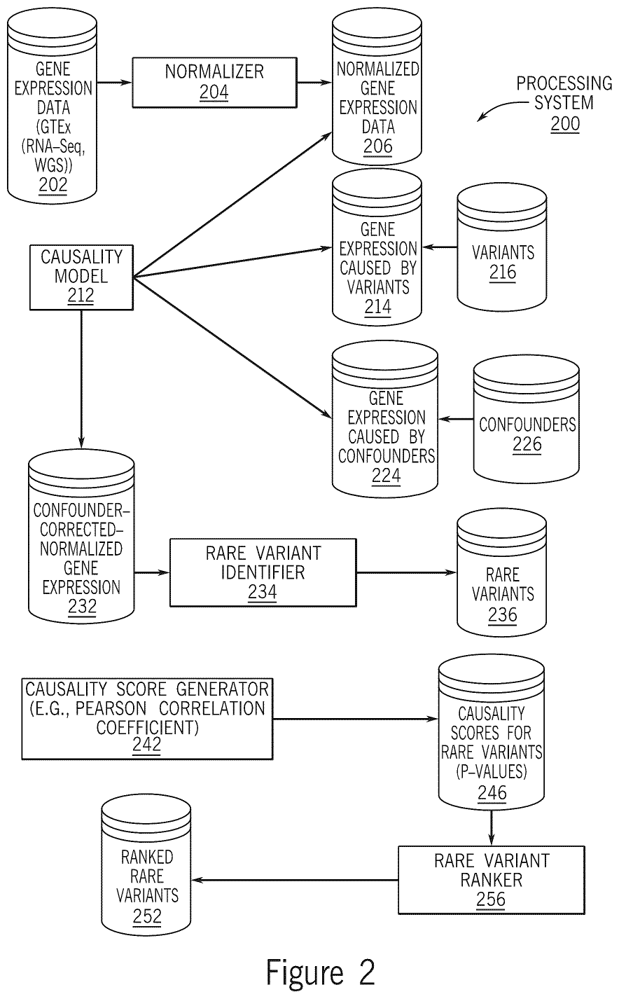

FIG. 2 illustrates one implementation of a processing system 200 that generates causality scores for outlier variants that cause extreme levels of gene expression. Datastore 202 comprises gene expression data, sourced, for example, from GTEx, RNA-Seq, or Whole genome sequencing (WGS). A normalizer 204 normalizes the gene expression data and stores the normalized gene expression data in a datastore 206. The normalized gene expression data can be measured by a mean and one or more standard deviations from the mean. In one implementation, the extreme levels of gene expression are determined from tail quantiles of the normalized gene expression data.

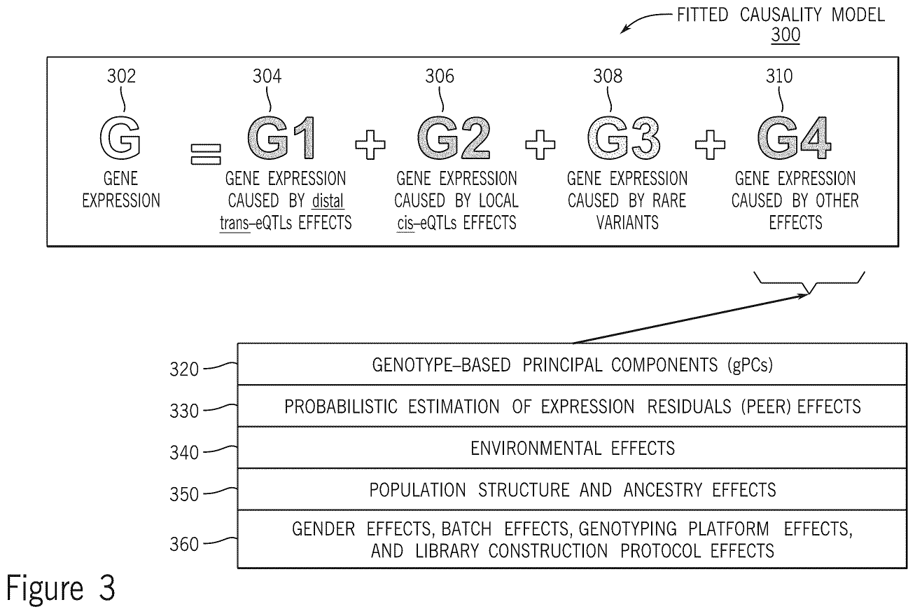

A causality model 212 is fitted to determine causal relationships between variants 216 and extreme levels of gene expression while controlling for a plurality of confounders 226. In one implementation, the fitted causality model 212 determines the causal relationships by predicting a particular gene expression level of a particular gene in a particular chromosome in dependence upon a variant-driven gene expression level caused by a particular variant, which is referred to herein as gene expression caused by variants 214. In one implementation, the fitted causality model 212 measures a contribution of the variant-driven gene expression level 214 (caused by the variants 216) as a variant effect covariate (illustrated later in FIG. 3 as 308). In one implementation, the fitted causality model 212 measures a contribution of the confounders-driven gene expression level 224 (caused by the confounders 226) as a plurality of confounder effect covariates (illustrated later in FIGS. 3 as 304, 306, and 310).

Examples of the causality model 212 include a logistic regression model, a linear regression model, an analysis of covariance (ANCOVA) model, and/or a multivariate analysis of covariance (MANCOVA) model. Examples of the confounders 226 include distal trans-expression quantitative trait loci (eQTLs) effects, local cis-eQTLs effects, genotype-based principal components (gPCs), expression residuals (PEER) effects, environmental effects, population structure and ancestry effects, and gender effects, batch effects, genotyping platform effects, and library construction protocol effects.

The causality model 212 generates, as output, confounder-corrected-normalized gene expression 232 caused by the variants 216 with the effects of the confounders 226 regressed out.

A rare variant identifier 234 identifies rare variants 236 from among the variants 216. The rare variants 216 can be selected based on an allele frequency cutoff. For example, the rare variants 236 can have a minor allele frequency (MAF) of less than 0.1%.

A causality score generator 242 generates causality scores 246 for the rare variants 236 based on the confounder-corrected-normalized gene expression 232. A particular causality score of a particular rare variant indicates a likelihood of the particular rare variant causing an extreme level of gene expression in those outlier individuals whose gene sequences contain the particular rare variant. In one implementation, the causality scores 246 are probability values (p-values). In one implementation, the p-values are determined by a Pearson correlation coefficient.

A rare variants ranker 256 ranks the rare variants 236 based on the causality scores 246 and stores the ranked rare variants in datastore 252.

### Causality Model

FIG. 3 depicts one implementation of a fitted causality model 300 that determines causal relationships between the rare variants and the extreme levels of gene expression while controlling for a plurality of confounders. The fitted causality model 300 has a dependent variable 302 titled “G” (gene expression) and a plurality of independent variables 304, 306, 308, and 310 respectively titled:
“G1” (gene expression caused by distal trans-eQTLs effects),
“G2” (gene expression caused by local cis-eQTLs effects),
“G3” (gene expression caused by the rare variants), and
“G4” (gene expression caused by other effects like genotype-based principal components (gPCs) 320, expression residuals (PEER) effects 330, environmental effects 340, population structure and ancestry effects 350, and gender effects, batch effects, genotyping platform effects, and library construction protocol effects 360).

The technology disclosed removes G1, G2, and G4 from G, i.e., G-G1-G2-G4=G3, to eliminate (or correct for) the confounding effects and to accurately determine the gene expression caused by the rare variants.

The fitted causality model 300 controls for the distal trans-eQTLs effects by predicting the particular gene expression level (G) 302 in dependence upon a trans gene expression level (G1) 304 caused by other genes in other chromosomes. In one implementation, the fitted causality model 300 measures a contribution of the trans gene expression level as a trans effect covariate.

The fitted causality model 300 controls for the local cis-eQTLs effects by predicting the particular gene expression level (G) 302 in dependence upon a cis gene expression level (G2) 306 caused by a presence of a plurality of common variants in a neighborhood of the particular gene. In some implementations, the neighborhood is defined by an offset from a transcription start site (TSS) in the particular gene. In one implementation, the fitted causality model 300 measures a contribution of the cis gene expression level as a cis effect covariate.

The fitted causality model 300 controls for the population structure and ancestry effects by predicting the particular gene expression level (G) 302 in dependence upon a gPC gene expression level (G4) 310 caused by the gPCs. In one implementation, the fitted causality model 300 measures a contribution of the gPC gene expression level (G4) 310 as a population structure and ancestry effect covariate.

The fitted causality model 300 controls for the PEER effects by predicting the particular gene expression level (G) 302 in dependence upon a PEER gene expression level (G4) 310 caused by the PEER. In one implementation, the fitted causality model 300 measures a contribution of the PEER gene expression level (G4) 310 as a PEER effect covariate.

The fitted causality model 300 controls for the environmental effects by predicting the particular gene expression level (G) 302 in dependence upon an environment gene expression level (G4) 310 caused by the environmental effects. In one implementation, the fitted causality model 300 measures a contribution of the environment gene expression level (G4) 310 as an environmental effect covariate.

In some implementations, the extreme levels of gene expression include over gene expression and under gene expression.

### Over Expression

In one implementation, the fitted causality model 300 determines the causal relationships between the rare variants and the over gene expression while controlling for the plurality of confounders. In some implementations, the fitted causality model 300 generates so-called “over causality scores” for the rare variants. A particular over causality score of the particular rare variant indicates a likelihood of the particular rare variant causing an over gene expression in those outlier individuals whose gene sequences contain the particular rare variant.

In one implementation, the over causality scores are over probability values (over p-values). In one implementation, the over p-values are determined by a Pearson correlation coefficient. In some implementations, the over p-values specify statistically unconfounded likelihoods of the rare variants increasing gene expression in genes that otherwise have lower gene expression relative to other genes in a gene set.

### Under Expression

In one implementation, the fitted causality model 300 determines the causal relationships between the rare variants and the under gene expression while controlling for the plurality of confounders. In some implementations, the fitted causality model 300 generates so-called “under causality scores” for the rare variants. A particular under causality score of the particular rare variant indicates a likelihood of the particular rare variant causing an under gene expression in those outlier individuals whose gene sequences contain the particular rare variant.

In one implementation, the under causality scores are under probability values (under p-values). In one implementation, the under p-values are determined by a Pearson correlation coefficient. In some implementations, the under p-values specify statistically unconfounded likelihoods of the rare variants decreasing gene expression in genes that otherwise have higher gene expression relative to other genes in a gene set.

In some implementations, the rare variants are non-coding variants. The non-coding variants can include five prime untranslated region (UTR) variants, three prime UTR variants, enhancer variants, and promoter variants.

In some implementations, the gene expression levels are further stratified into tissue-specific gene expression levels for a plurality of tissues. In one implementation, the gene expression levels for each gene in each tissue are normalized using quantile normalization. In some implementations, the causality model is fitted separately for each tissue. In some implementations, the causality model is fitted using stratification.

### Ranking

In some implementations, a ranking of the rare variants is generated based on the causality scores. In one implementation, a ranking of the rare variants is generated based on the over causality scores. In one implementation, a ranking of the rare variants is generated based on the under causality scores.

In some implementations, the rare variants are singleton variants. In one implementation, a singleton variant occurs in only one outlier individual from the outlier individuals.

### Causality Scores

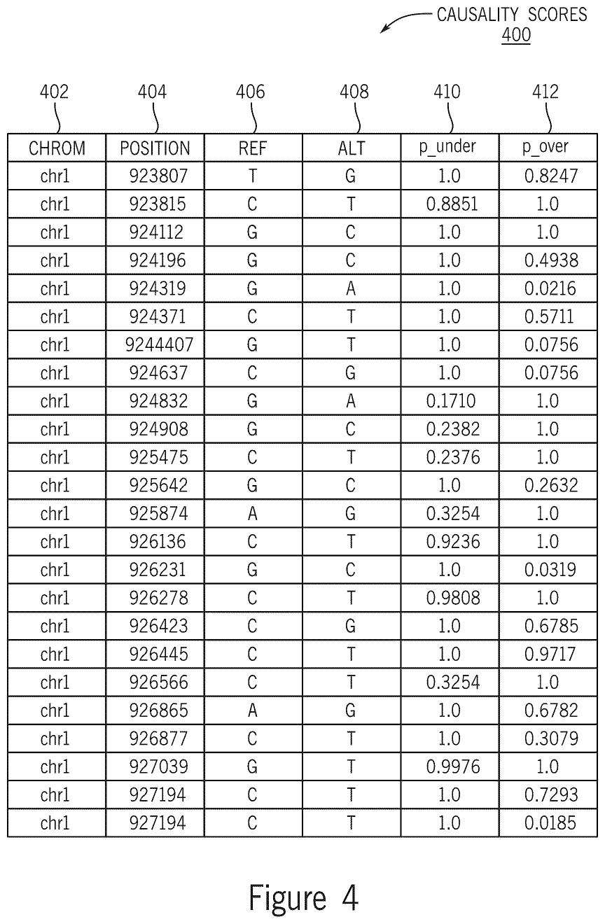

FIG. 4 shows examples of causality scores 400 generated by the technology disclosed for a sample of rare variants. FIG. 4 shows a “chrom” column 402 that identifies the chromosome on which the rare variants are located. FIG. 4 also shows a “position” column 404 that identifies the location of the rare variants. FIG. 4 also shows a “REF” column 406 that identifies the reference nucleotides (or bases) corresponding to the rare variants. FIG. 4 also shows an “ALT” column 408 that identifies the alternate nucleotides (or bases) representing the rare variants.

FIG. 4 also shows a “p_under” column 410 that identifies the under causality scores for the rare variants. The p_under values are inversely related to the under causality scores, i.e., the higher the p_under value, the lesser the likelihood of the rare variant causing under gene expression, and the lower the p_under value, the greater the likelihood of the rare variant causing under gene expression.

FIG. 4 also shows a “p_over” column 412 that identifies the over causality scores for the rare variants. The p_over values are inversely related to the over causality scores, i.e., the higher the p_over value, the lesser the likelihood of the rare variant causing over gene expression, and the lower the p_over value, the greater the likelihood of the rare variant causing over gene expression.

### Performance Results as Objective Indicia of Non-Obviousness and Inventiveness

Comparing and detecting differences between sample distributions and reference distributions, or sample outliers from reference distributions can include the use of parametric and non-parametric statistical testing such as the use of (one-or two-tailed) t-tests, Mann-Whitney Rank Sum test and others, including the use of a z-score, such as a Median Absolute Deviation based z-score (e.g., such as used by Stumm et al 2014, Prenat Diagn 34:185). When comparing a distribution to (or outliers from) a reference distribution, then in certain implementations, the comparison is distinguished (and/or identified as being significantly different) if the separation of the means, medians, or individual samples are greater than about 1.5, 1.6, 1.7, 1.8, 1.9, 1.95, 1.97, 2.0, or greater than about 2.0 standard distributions (“SD”) of the reference distribution; and/or if an individual sample separates from the reference distribution with a z-score of greater than about 1.5, 1.6, 1.7, 1.8, 1.9, 2.0, 2.5, 2.7, 2.8, 2.9, 3.0, 3.1, 3.2, 3.3, 3.4, 3.5, 3.75, 4.0, 4.5, 5.0 or greater than about 5.0.

In certain implementations, a parameter (such as a mean, median, standard deviation, median absolute deviation, or z-score) is calculated in respect of a set of samples. In certain of such implementations, such a calculated parameter is used to identify outliers from those test samples detected/analyzed. In particular implementations, such a parameter is calculated from all test samples without knowledge of the identity of any outliers (e.g., a “masked” analysis). In other particular implementations, such a parameter is calculated from a set of reference samples know to be (non-outlying) standards or test samples that are presumed to be (or are unlikely to be) such standards.

In certain implementations, in the context of a data set a z-score (or an equivalent statistic based on the distribution pattern of replicates of a parameter) can be calculated to identify an outlying data point(s) (for example, representing an extreme level of gene expression (under or over)), the data representing such data point removed from the data set and a subsequent z-score analysis be conducted on the data set to seek to identify further outliers. Such an iterative z-score analysis may be particular helpful sometimes when two or more samples may skew a single z-score analysis, and/or where follow-up tests are available to confirm false positives and hence avoiding false negatives is potentially more important that the (initial) identification of false positives.

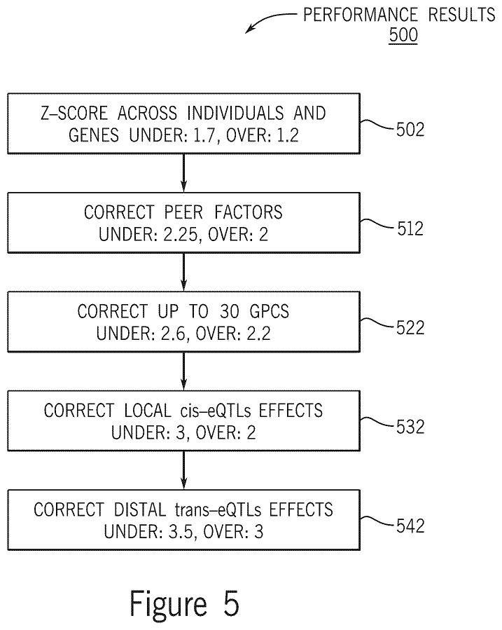

FIG. 5 shows that performance results 500, measured as a z-score across individuals and genes, improve progressively by successively correcting for the plurality of confounders using the fitted causality model 300. In particular, an under z-score is determined to measure the correlation between the rare variants and under gene expression, and an over z-score is determined to measure the correlation between the rare variants and over gene expression.

At 502, the under z-score is 1.7, and the over z-score is 1.2. At this stage, none of the confounders are corrected.

At 512, the under z-score increases to 2.25, and the over z-score increases to 2. At this stage, the PEER factors are corrected.

At 522, the under z-score increases to 2.6, and the over z-score increases to 2.2. At this stage, 30 gPCs are corrected.

At 532, the under z-score increases to 3, and the over z-score decreases to 2. At this stage, the local cis-eQTLs effects are corrected.

At 542, the under z-score increases to 3.5, and the over z-score increases to 3. At this stage, the distal trans-eQTLs effects are corrected.

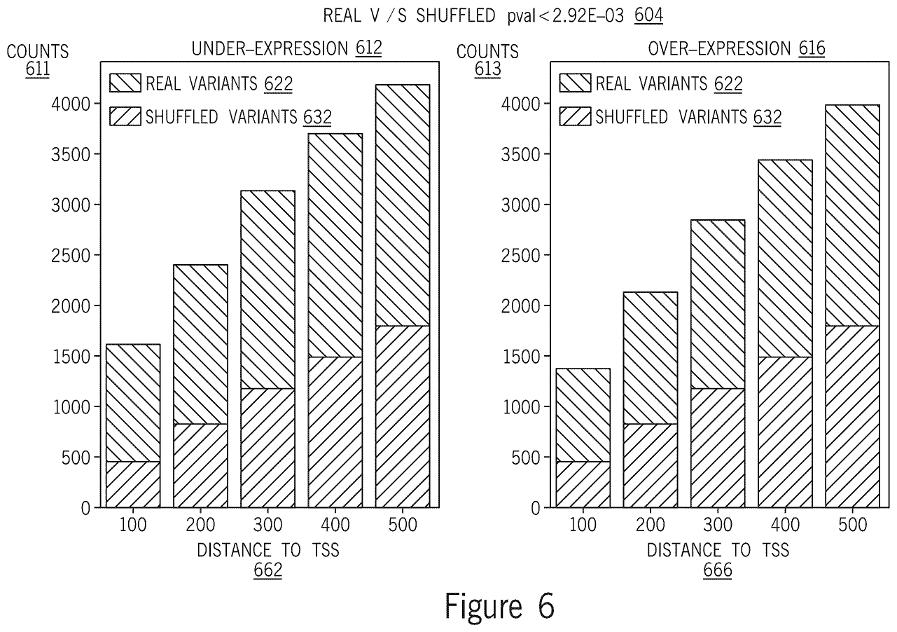

FIG. 6 compares the counts 611, 613 of real rare variants 622 and shuffled (or randomly selected) variants 632 that are identified as causing under gene expression 612 and over gene expression 616 for a given p-value 604 from the fitted causality model 300. As shown in FIG. 6, the count-based correlations for the real rare variants 622 are consistently higher than the shuffled variants 632. In FIG. 6, the x-axis is the distance 662, 666 of the real rare variants 622 and the shuffled variants 632 from the transcription start site (TSS). In FIG. 6, the y-axis is the count 611, 613 of the real rare variants 622 and the shuffled variants 632.

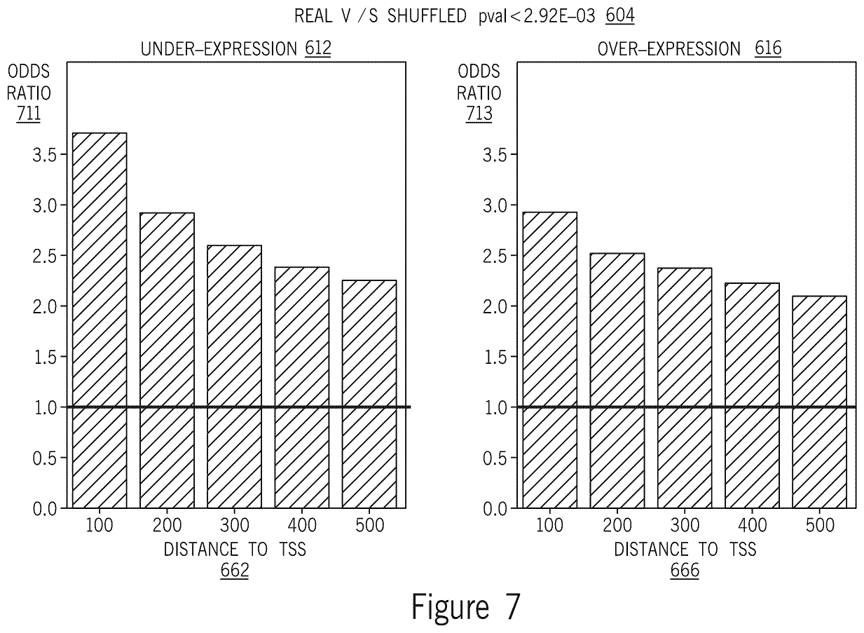

FIG. 7 shows the odds ratios 711, 713 that compare the causality of the real rare variants 622 and the shuffled variants 632 with respect to under gene expression 612 and over gene expression 616 for the given p-value 604 from the fitted causality model 300. As shown in FIG. 7, the consistently high odds ratios 711, 713 for the different TSS distances 662, 666 demonstrate strong causality of the real rare variants 622.

### Base Resolution Evolutionary Conservation Prediction

### Chromatin Model

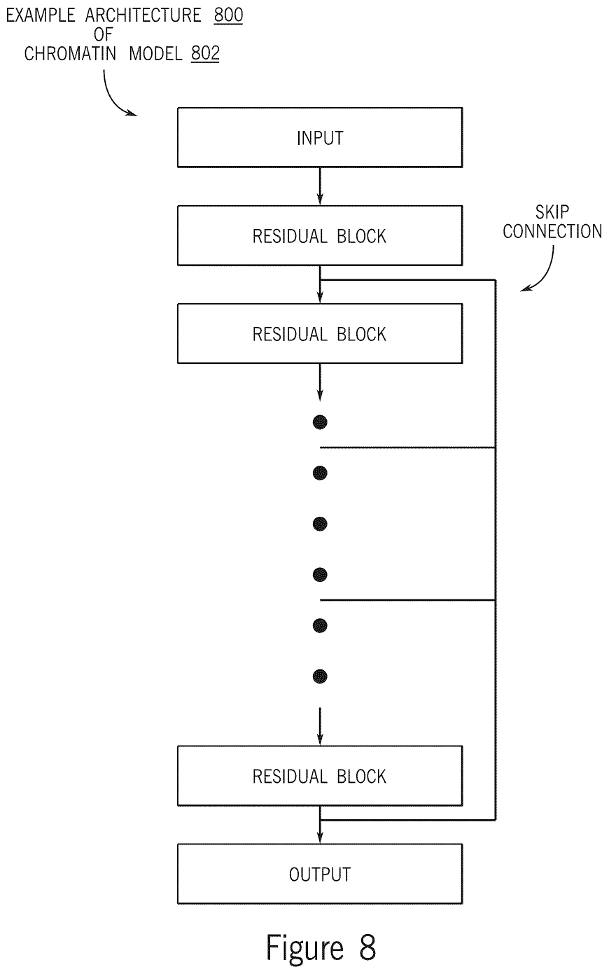

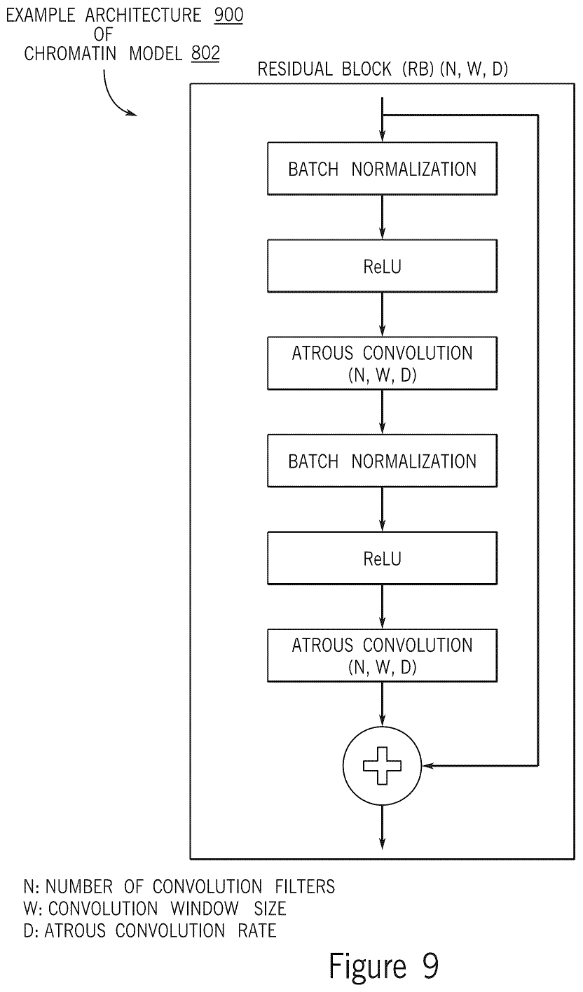

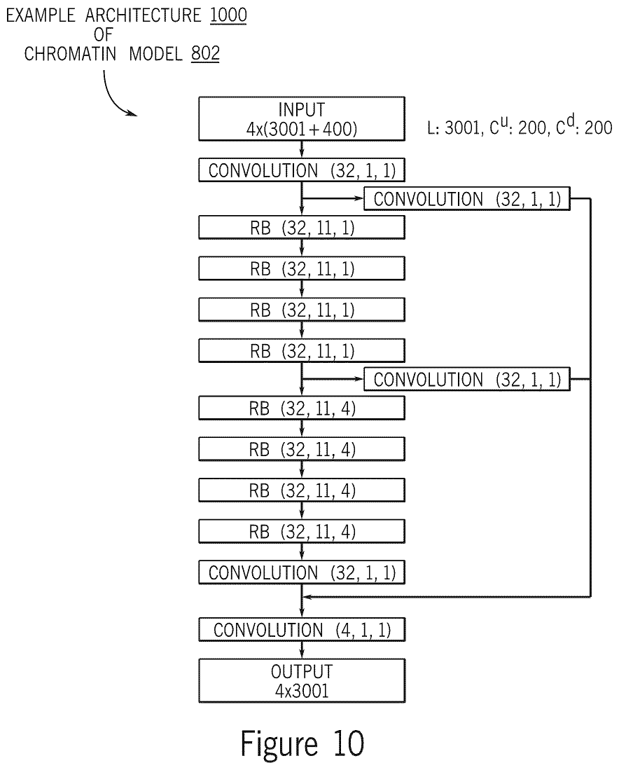

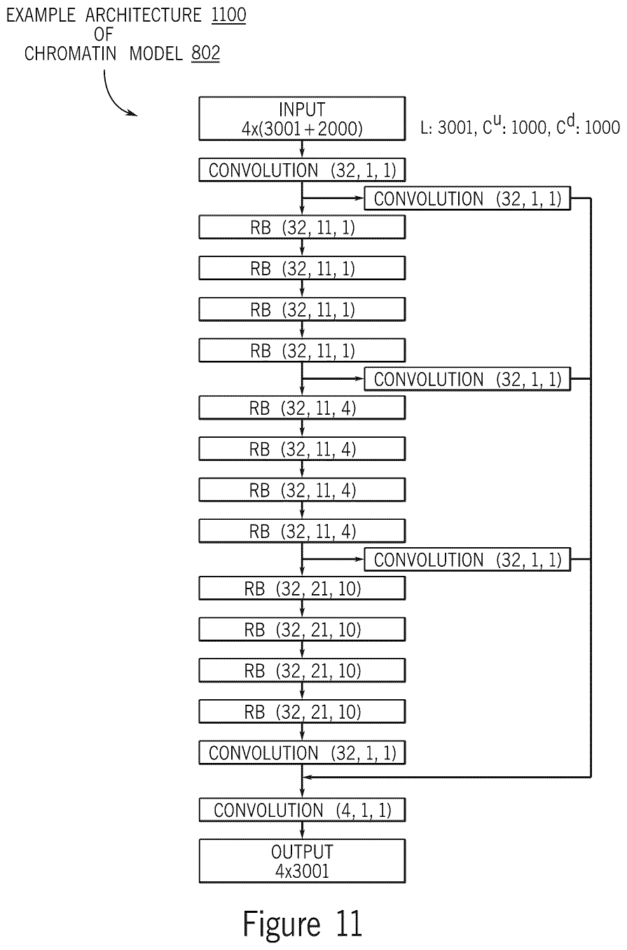

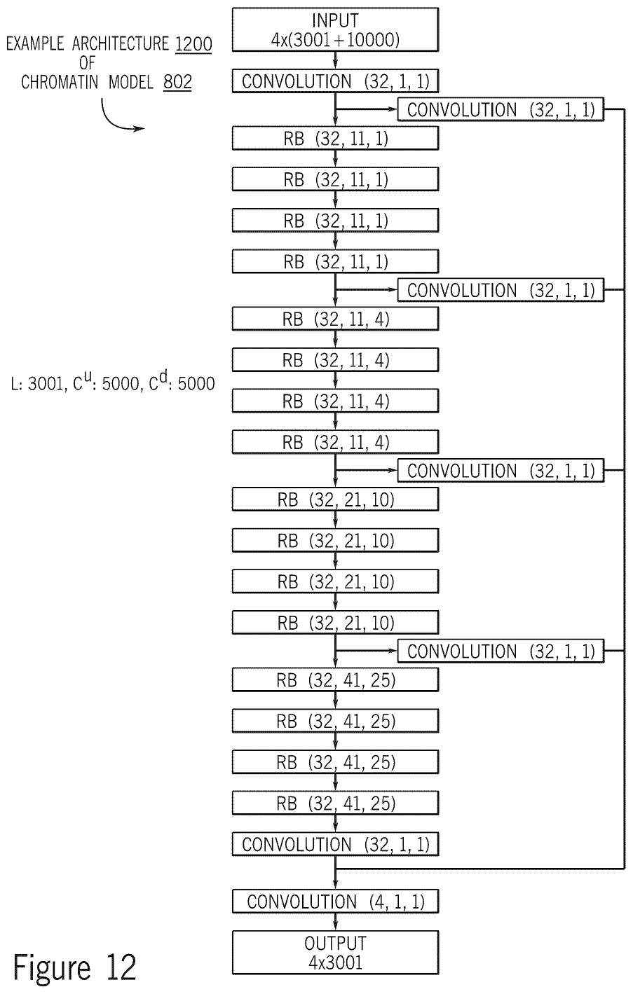

FIG. 8 shows a first example architecture 800 of the disclosed chromatin model 802. FIG. 9 shows a second example architecture 900 of the disclosed chromatin model 802. FIG. 10 shows a third example architecture 1000 of the disclosed chromatin model 802. FIG. 11 shows a fourth example architecture 1100 of the disclosed chromatin model 802. FIG. 12 shows a fifth example architecture 1200 of the disclosed chromatin model 802.

As shown in FIG. 8, the chromatin model 802 contains groups of residual blocks arranged in a sequence from lowest to highest. Each group of residual blocks is parameterized by a number of convolution filters in the residual blocks, a convolution window size of the residual blocks, and an atrous convolution rate of the residual blocks. The atrous convolution rate progresses non-exponentially from a lower residual block group to a higher residual block group, in some implementations. In other implementations, it progresses exponentially. The size of convolution window varies between groups of residual blocks, and each residual block comprises at least one batch normalization layer, at least one rectified linear unit (abbreviated ReLU) layer, at least one atrous convolution layer, and at least one residual connection.

As shown in FIG. 9, the dimensionality of the input is (Cu+L+Cd)×4, where Cu is a number of upstream flanking context bases, Cd is a number of downstream flanking context bases, and L is a number of bases in the input promoter sequence. The dimensionality of the output is 4×L. In some implementations, each group of residual blocks produces an intermediate output by processing a preceding input and the dimensionality of the intermediate output is (I-[{(W−1)*D}*A])×N, where I is dimensionality of the preceding input, W is convolution window size of the residual blocks, D is atrous convolution rate of the residual blocks, A is a number of atrous convolution layers in the group, and N is a number of convolution filters in the residual blocks.

Example architecture 1000 is used when the input has 200 upstream flanking context bases (Cu) to the left of the input sequence and 200 downstream flanking context bases (Cd) to the right of the input sequence. The length of the input sequence (L) can be arbitrary, such as 3001. In example architecture 1000, each residual block in a first group has 32 convolution filters, 11 convolution window size, and 1 atrous convolution rate and each residual block in a second group has 32 convolution filters, 11 convolution window size, and 4 atrous convolution rate. In other architectures, each residual block has 32 convolution filters, 11 convolution window size, and 1 atrous convolution rate.

Example architecture 1100 is used when the input has one thousand upstream flanking context bases (Cu) to the left of the input sequence and one thousand downstream flanking context bases (Cd) to the right of the input sequence. The length of the input sequence (L) can be arbitrary, such as 3001. In example architecture 1100, there are at least three groups of four residual blocks and at least three skip connections. Each residual block in a first group has 32 convolution filters, 11 convolution window size, and 1 atrous convolution rate, each residual block in a second group has 32 convolution filters, 11 convolution window size, and 4 atrous convolution rate, and each residual block in a third group has 32 convolution filters, 21 convolution window size, and 19 atrous convolution rate.

Example architecture 1200 is used when the input has five thousand upstream flanking context bases (Cu) to the left of the input sequence and five thousand downstream flanking context bases (Cd) to the right of the input sequence. The length of the input sequence (L) can be arbitrary, such as 3001. In example architecture 1200, there are at least four groups of four residual blocks and at least four skip connections. Each residual block in a first group has 32 convolution filters, 11 convolution window size, and 1 atrous convolution rate, each residual block in a second group has 32 convolution filters, 11 convolution window size, and 4 atrous convolution rate, each residual block in a third group has 32 convolution filters, 21 convolution window size, and 19 atrous convolution rate, and each residual block in a fourth group has 32 convolution filters, 41 convolution window size, and 25 atrous convolution rate.

Generally speaking, the chromatin model 802 can be a rule-based model, a tree-based model, or a machine learning model. Examples include a multilayer perceptron (MLP), a feedforward neural network, a fully-connected neural network, a fully convolution neural network, a ResNet, a sequence-to-sequence (Seq2Seq) model like WaveNet, a semantic segmentation neural network, and a generative adversarial network (GAN) (e.g., CycleGAN, StyleGAN, pixelRNN, text-2-image, DiscoGAN, IsGAN).

In some implementations, the chromatin model 802 can include self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, BERT, SpanBERT, ROBERTa, XLNet, ELECTRA, UniLM, BART, T5, ERNIE (THU), KnowBERT, DeiT-Ti, DeiT-S, DeiT-B, T2T-ViT-14, T2T-VIT-19,T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DC5, TSP-FCOS, TSP-RCNN, ACT+MKDD (L=32), ACT+MKDD (L=16), SMCA, Efficient DETR, UP-DETR, UP-DETR, ViTB/16-FRCNN, ViT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

In some implementations, examples of the chromatin model 802 include a convolution neural network (CNN) with a plurality of convolution layers, a recurrent neural network (RNN) such as a long short-term memory network (LSTM), bi-directional LSTM (Bi-LSTM), or a gated recurrent unit, and a combination of both a CNN and an RNN.

In some implementations, the chromatin model 802 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The chromatin model 802 can use one or more loss functions such as logistic regression/log loss, multi-class cross-entropy/softmax loss, binary cross-entropy loss, mean-squared error loss, L1 loss, L2 loss, smooth L1 loss, and Huber loss. The chromatin model 802 can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The chromatin model 802 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

In some implementations, the chromatin model 802 can be a linear regression model, a logistic regression model, an Elastic Net model, a support vector machine (SVM), a random forest (RF), a decision tree, or a boosted decision tree (e.g., XGBoost), or some other tree-based logic (e.g., metric tree, kd-tree, R-tree, universal B-tree, X-tree, ball tree, locality sensitive hash, and inverted index). The chromatin model 802 can be an ensemble of multiple models, in some implementations.

In some implementations, the chromatin model 802 can be trained using backpropagation-based gradient update techniques. Example gradient descent techniques that can be used for training the models include stochastic gradient descent, batch gradient descent, and mini-batch gradient descent. Some examples of gradient descent optimization algorithms that can be used to train the models are Momentum, Nesterov accelerated gradient, Adagrad, Adadelta, RMSprop, Adam, AdaMax, Nadam, and AMSGrad.

### Input Base Sequence

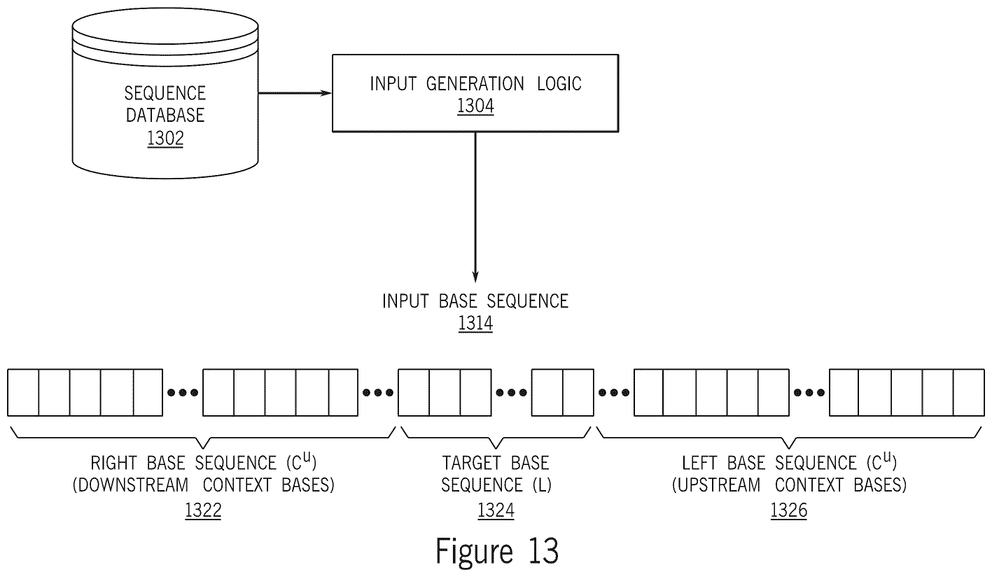

FIG. 13 illustrates an input generation logic 1304 that accesses a sequence database 1302 (e.g., GTEx, RNA-Seq, WGS) and generates an input base sequence 1314. The input base sequence 1314 includes a target base sequence 1324. The target base sequence 1324 is flanked by a right base sequence 1322 with downstream context bases, and a left base sequence 1326 with upstream context bases.

### Base Resolution Output sequence

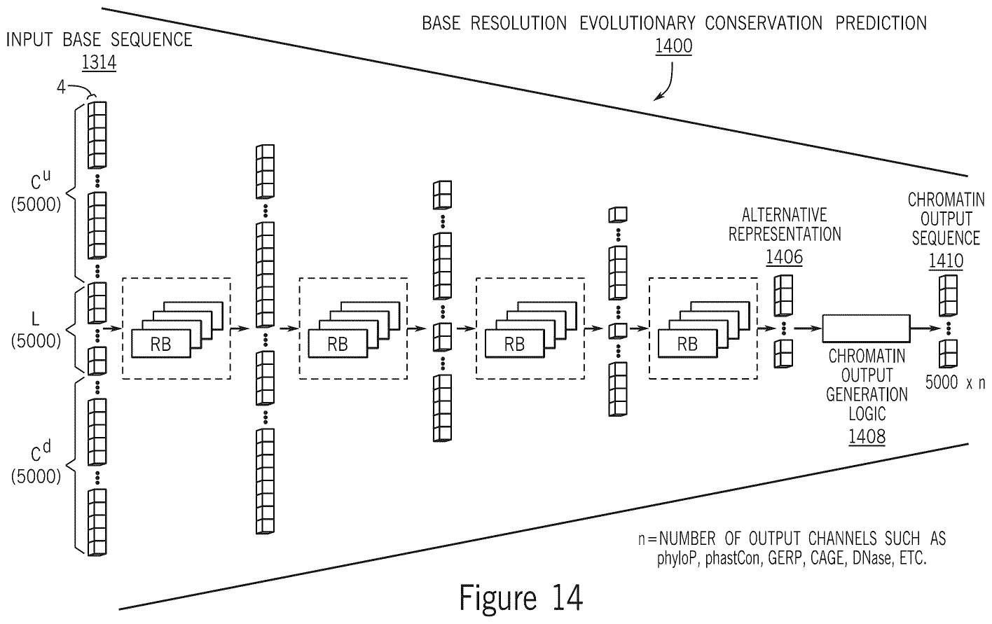

FIG. 14 depicts one implementation of base resolution evolutionary conservation prediction 1400 by the chromatin model 802. The chromatin model 802 processes the input base sequence 1314 and generates an alternative representation 1406 of the input base sequence 1314. In one implementation, the alternative representation 1406 is a convolved representation of the input base sequence 1314 when the input base sequence 1314 is processed by a cascade of convolution layers of the chromatin model 802.

A chromatin output generation logic 1408 processes the alternative representation 1406 of the input base sequence 1314 and generates a output sequence 1410 of respective per-base chromatin outputs for respective target bases in the target base sequence 1324.

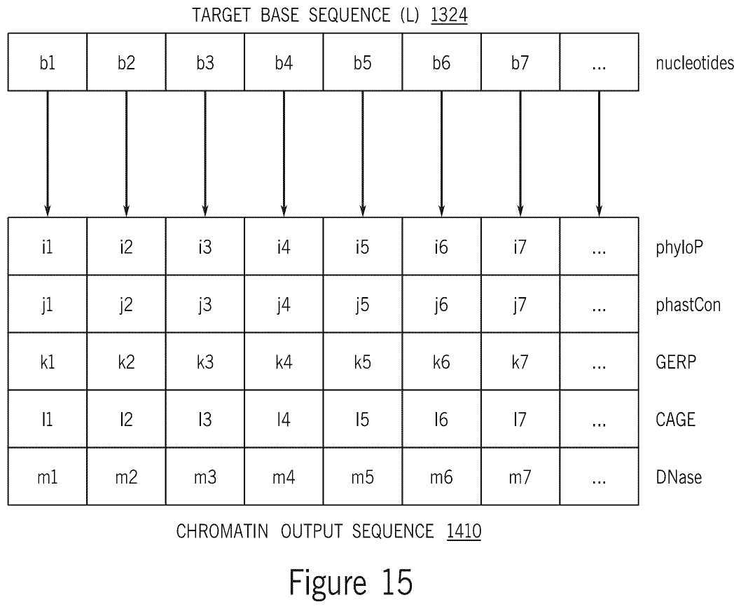

FIG. 15 shows an example of the output sequence 1410 corresponding to the target base sequence 1324. A given per-base chromatin output in the output sequence 1410 for a given target base at a given position in the target base sequence 1324 specifies a measure of evolutionary conservation of the given target base across a plurality of species. The plurality of species can include homologous species.

Predicting functional consequences of variants relies at least in part on the assumption that crucial amino acids for protein families are conserved through evolution due to negative selection (i.e., amino acid changes at these sites were deleterious in the past), and that mutations at these sites have an increased likelihood of being pathogenic (causing disease) in humans. Homologous sequences of a target protein are collected and aligned in a multiple sequence alignment (MSA). A metric of conservation is computed based on weighted scores and/or frequencies of different amino acids observed in the target position in the MSA.

An MSA is generally the alignment of three or more biological sequences, protein, or nucleic acid, of similar length. From the alignment, the degree of homology can be inferred and the evolutionary relationships among the sequences studied. An MSA is also a tool used to identify the evolutionary relationships and common patterns among genes. Alignments are generated and analyzed using computational algorithms. Dynamic and heuristic approaches are used in most MSA algorithms. One of the objectives of alignment is to detect structural or functional identities and similarities between residues in protein sequences relative to other protein sequences.

Homolog information pertaining to aligned sequences in the MSA can be represented by two matrices (evolutionary conservation metrics): a position-specific scoring matrix (PSSM) and a position-specific frequency matrix (PSFM). PSSMs and PSFMs reflect the conservation of residues at specific positions of protein chains based on evolutionary information.

In some implementations, the given per-base chromatin output further specifies a measure of transcription initiation of the given target base at the given position.

In one implementation, the measure of evolutionary conservation is a phylogenetic P-values (phyloP) score that specifies a deviation from a null model of neural substitution to detect a reduction in a rate of substitution of the given target base at the given position as conservation, and to detect an increase in the rate of substitution of the given target base at the given position as acceleration.

In one implementation, the measure of evolutionary conservation is a phastCons score that specifies a posterior probability of the given target base at the given position having a conserved state or a non-conserved state.

In one implementation, the measure of evolutionary conservation is a genomic evolutionary rate profiling (GERP) score that specifies a reduction in a number of substitutions of the given target base at the given position across the plurality of species.

In one implementation, the measure of transcription initiation is a cap analysis of gene expression (CAGE) score that specifies a transcription initiation frequency of the given target base at the given position.

In some implementations, the given per-base chromatin output further specifies a confounder signal level for the given target base at the given position. In one implementation, the confounder signal level specifies DNase I-hypersensitive sites (DHSs). In one implementation, the confounder signal level specifies assay for transposase-accessible chromatin with sequencing (ATAC-Seq). In another implementation, the confounder signal level specifies transcription factor (TF) bindings. In yet another implementation, the confounder signal level specifies histone modification (HM) marks. In yet further implementation, the confounder signal level specifies DNA methylation marks.

### Base Resolution Gene Expression Level Prediction

### Gene Expression Model

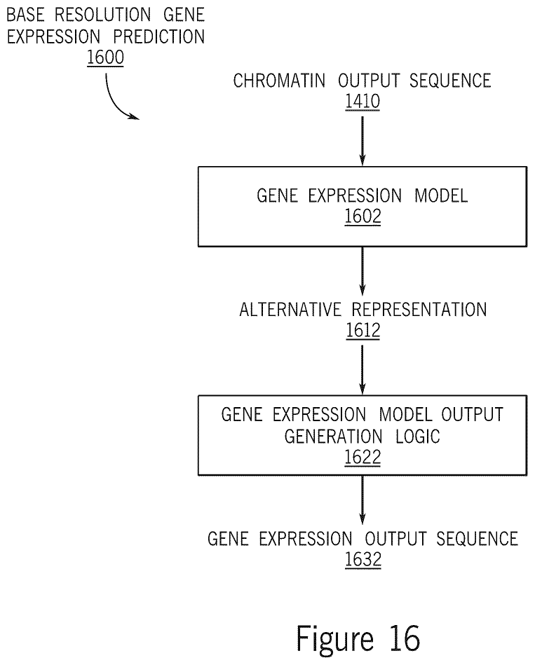

FIG. 16 shows one implementation of the disclosed gene expression model 1600. Generally speaking, the gene expression model 1602 can be a rule-based model, a tree-based model, or a machine learning model. Examples include a multilayer perceptron (MLP), a feedforward neural network, a fully-connected neural network, a fully convolution neural network, a ResNet, a sequence-to-sequence (Seq2Seq) model like WaveNet, a semantic segmentation neural network, and a generative adversarial network (GAN) (e.g., CycleGAN, StyleGAN, pixelRNN, text-2-image, DiscoGAN, IsGAN).

In some implementations, the gene expression model 1602 can include self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, BERT, SpanBERT, ROBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DC5, TSP-FCOS, TSP-RCNN, ACT+MKDD (L=32), ACT+MKDD (L=16), SMCA, Efficient DETR, UP-DETR, UP-DETR, VITB/16-FRCNN, VIT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

In some implementations, examples of the gene expression model 1602 include a convolution neural network (CNN) with a plurality of convolution layers, a recurrent neural network (RNN) such as a long short-term memory network (LSTM), bi-directional LSTM (Bi-LSTM), or a gated recurrent unit, and a combination of both a CNN and an RNN.

In some implementations, the gene expression model 1602 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The gene expression model 1602 can use one or more loss functions such as logistic regression/log loss, multi-class cross-entropy/softmax loss, binary cross-entropy loss, mean-squared error loss, L1 loss, L2 loss, smooth L1 loss, and Huber loss. The gene expression model 1602 can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The gene expression model 1602 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

In some implementations, the gene expression model 1602 can be a linear regression model, a logistic regression model, an Elastic Net model, a support vector machine (SVM), a random forest (RF), a decision tree, or a boosted decision tree (e.g., XGBoost), or some other tree-based logic (e.g., metric tree, kd-tree, R-tree, universal B-tree, X-tree, ball tree, locality sensitive hash, and inverted index). The gene expression model 1602 can be an ensemble of multiple models, in some implementations.

In some implementations, the gene expression model 1602 can be trained using backpropagation-based gradient update techniques. Example gradient descent techniques that can be used for training the models include stochastic gradient descent, batch gradient descent, and mini-batch gradient descent. Some examples of gradient descent optimization algorithms that can be used to train the models are Momentum, Nesterov accelerated gradient, Adagrad, Adadelta, RMSprop, Adam, AdaMax, Nadam, and AMSGrad.

The gene expression model 1602 processes the output sequence 1410 and generates an alternative representation 1612 of the output sequence 1410. In one implementation, the alternative representation 1612 is a convolved representation of the output sequence 1410 when the output sequence 1410 is processed by a cascade of convolution layers of the gene expression model 1602.

A gene expression model output generation logic 1622 processes the alternative representation 1612 of the output sequence 1410 and generates a gene expression output sequence 1632 of respective per-base gene expression outputs for the respective target bases in the target base sequence 1324.

A given per-base gene expression output in the gene expression output sequence 1632 for the given target base at the given position specifies a measure of gene expression level of the given target base at the given position. In one implementation, the gene expression level is measured in a per-base metric such as CAGE transcription start site (CTSS). In another implementation, the gene expression level is measured in a per-gene metric such as transcripts per million (TPM) or reads per kilobase of transcript (RPKM). In yet another implementation, the gene expression level is measured in a per-gene metric such as fragments per kilobase million (FPKM).

### Variant Pathogenicity Classification

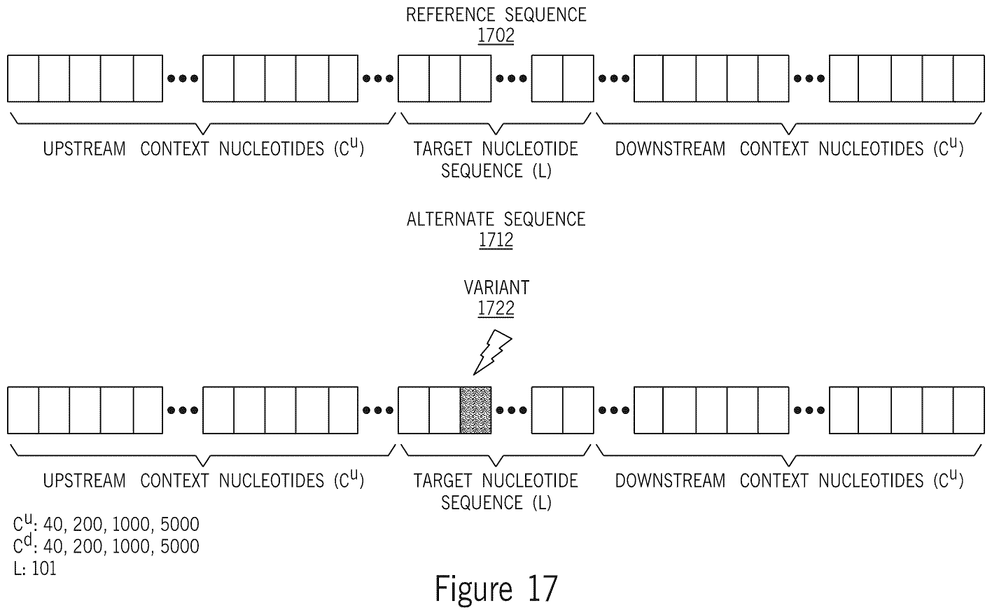

FIG. 17 shows an example of a reference sequence 1702 and an alternate sequence 1722 (or alternative sequence). The alternate sequence 1712 differs from the reference sequence 1702 by a variant nucleotide 1722.

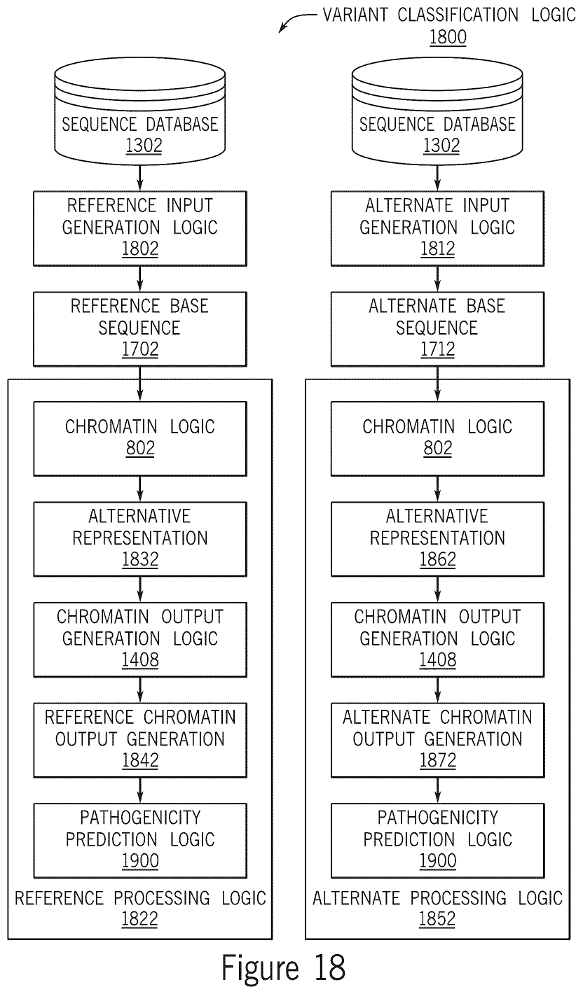

FIG. 18 illustrates one implementation of the disclosed variant classification logic 1800. The variant classification logic 1800 is further configured to comprise a reference input generation logic 1802 that accesses the sequence database 1302 and generates the reference base sequence 1702. The reference base sequence 1702 includes a reference target base sequence. The reference target base sequence includes a reference base at a position-under-analysis. The reference base is flanked by a right base sequence with downstream context bases, and a left base sequence with upstream context bases.

The variant classification logic 1800 is further configured to comprise an alternate input generation logic 1812 that accesses the sequence database 1302 and generates the alternate base sequence 1712. The alternate base sequence 1712 includes an alternate target base sequence. The alternate target base sequence includes the alternate base 1722 at the position-under-analysis. The alternate base 1722 is flanked by the right base sequence with the downstream context bases, and the left base sequence with the upstream context bases.

The variant classification logic 1800 is further configured to comprise a reference processing logic 1822 that causes the chromatin model 802 to process the reference base sequence 1702 and generate an alternative representation 1832 of the reference base sequence 1702, and further causes the chromatin output generation logic 1408 to process the alternative representation 1832 of the reference base sequence 1702 and generate a reference output sequence 1842 of respective per-base reference chromatin outputs for respective reference target bases in the reference target base sequence.

A given per-base reference chromatin output in the reference output sequence 1842 for a given reference target base at a given position in the reference target base sequence specifies a measure of evolutionary conservation of the given reference target base across the plurality of species.

The variant classification logic 1800 is further configured to comprise an alternate processing logic 1852 that causes the chromatin model 802 to process the alternate base sequence 1712 and generate an alternative representation 1862 of the alternate base sequence 1712, and further causes the chromatin output generation logic 1408 to process the alternative representation 1862 of the alternate base sequence 1712 and generate an alternate output sequence 1872 of respective per-base alternate chromatin outputs for respective alternate target bases in the alternate target base sequence.

A given per-base alternate chromatin output in the alternate output sequence 1872 for a given alternate target base at a given position in the alternate target base sequence specifies a measure of evolutionary conservation of the given alternate target base across the plurality of species.

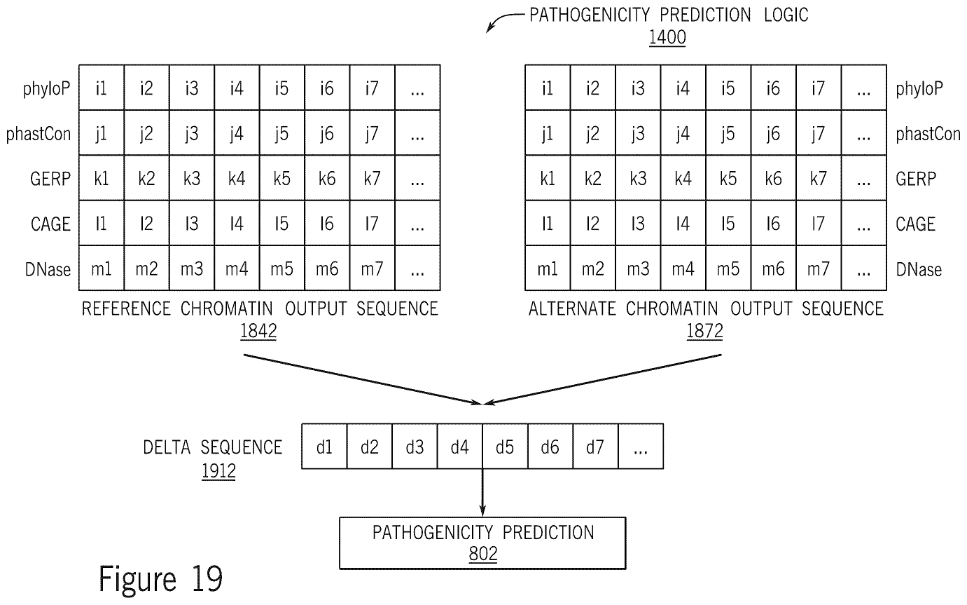

FIG. 19 illustrates one implementation of the disclosed pathogenicity prediction logic 1900. The variant classification logic 1800 is further configured to comprise the pathogenicity prediction logic 1900 that position-wise compares the reference output sequence 1842 and the alternate output sequence 1872 and generates a delta sequence 1912 with position-wise sequence diffs for positions in the reference output sequence 1842 and the alternate output sequence 1872.

The pathogenicity prediction logic 1900 is further configured to generate a pathogenicity prediction 1922 for the alternate base 1722 in dependence upon the delta sequence 1912. In one implementation, the pathogenicity prediction logic 1900 is further configured to accumulate the position-wise sequence diffs into an accumulated sequence value and generate the pathogenicity prediction 1922 for the alternate base 1722 in dependence upon the accumulated sequence value. In some implementations, the accumulated sequence value is an average or max of the position-wise sequence diffs. In other implementations, the accumulated sequence value is a sum of the position-wise sequence diffs.

In some implementations, the pathogenicity prediction logic 1900 is further configured to position-wise compare respective portions of the reference output sequence 1842 and the alternate output sequence 1872 and generate a delta sub-sequence with position-wise sub-sequence diffs for positions in the respective portions.

In one implementation, the respective portions span right and left flanking positions around the position-under-analysis. In some implementations, the pathogenicity prediction logic 1900 is further configured to generate a pathogenicity prediction for the alternate base 1722 in dependence upon the delta sub-sequence. In one implementation, the pathogenicity prediction can be a score between zero and one, where zero represents absolute benignness and one represents absolute pathogenicity. In other implementations, a cutoff can be used, such as a pathogenicity score above five, for example, can be considered pathogenic, and below five can be considered benign.

In some implementations, the pathogenicity prediction logic 1900 is further configured to accumulate the position-wise sub-sequence diffs into an accumulated sub-sequence value and generate the pathogenicity prediction for the alternate base 1722 in dependence upon the accumulated sub-sequence value. In one implementation, the accumulated sub-sequence value is an average of the position-wise sub-sequence diffs. In another implementation, the accumulated sub-sequence value is a sum or max of the position-wise sub-sequence diffs.

### Computer System

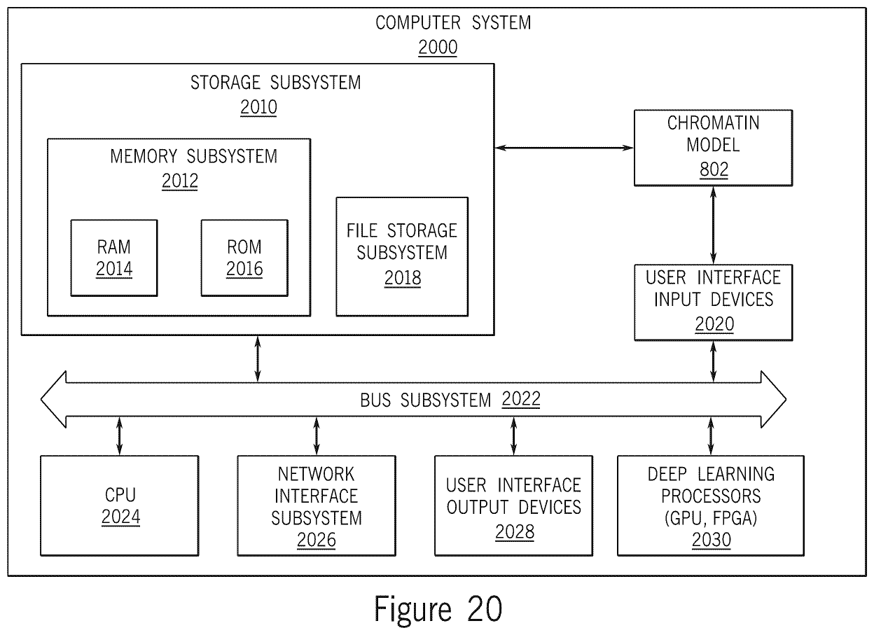

FIG. 20 is an example computer system 2000 that can be used to implement various aspects of the technology disclosed. Computer system 2000 includes at least one central processing unit (CPU) 2024 that communicates with a number of peripheral devices via bus subsystem 2022. These peripheral devices can include a storage subsystem 2010 including, for example, memory devices and a file storage subsystem 2018, user interface input devices 2020, user interface output devices 2028, and a network interface subsystem 2026. The input and output devices allow user interaction with computer system 2000. Network interface subsystem 2026 provides an interface to outside networks, including an interface to corresponding interface devices in other computer systems.

In one implementation, the chromatin model 802 is communicably linked to the storage subsystem 2010 and the user interface input devices 2020.

User interface input devices 2020 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 2000.

User interface output devices 2028 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 2000 to the user or to another machine or computer system.

Storage subsystem 2010 stores programming and data constructs that provide the functionality of some or all of the modules and methods described herein. These software modules are generally executed by processors 2030.

Processors 2030 can be graphics processing units (GPUs), field-programmable gate arrays (FPGAs), application-specific integrated circuits (ASICs), and/or coarse-grained reconfigurable architectures (CGRAs). Processors 2030 can be hosted by a deep learning cloud platform such as Google Cloud Platform™, Xilinx™, and Cirrascale™. Examples of processors 2078 include Google's Tensor Processing Unit (TPU)™, rackmount solutions like GX4 Rackmount Series™, GX20 Rackmount Series™, NVIDIA DGX-1™, Microsoft'Stratix V FPGA™, Graphcore's Intelligent Processor Unit (IPU)™, Qualcomm's Zeroth Platform™ with Snapdragon processors™, NVIDIA's Volta™, NVIDIA's DRIVE PXT™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 2012 used in the storage subsystem 2010 can include a number of memories including a main random access memory (RAM) 2014 for storage of instructions and data during program execution and a read only memory (ROM) 2016 in which fixed instructions are stored. A file storage subsystem 2018 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 2018 in the storage subsystem 2010, or in other machines accessible by the processor.

Bus subsystem 2022 provides a mechanism for letting the various components and subsystems of computer system 2000 communicate with each other as intended. Although bus subsystem 2022 is shown schematically as a single bus, alternative implementations of the bus subsystem can use multiple busses.

Computer system 2000 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 2000 depicted in FIG. 20 is intended only as a specific example for purposes of illustrating the preferred implementations of the present invention. Many other configurations of computer system 2000 are possible having more or less components than the computer system depicted in FIG. 20.

### Clauses

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections-these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

### Clauses Set 1

1. A computer-implemented method of identifying rare variants that cause extreme levels of gene expression, including:
   accessing gene expression levels for a group of individuals;
   normalizing the gene expression levels, and identifying those outlier individuals from the group of individuals that have extreme levels of gene expression, wherein the extreme levels of gene expression are determined from tail quantiles of the normalized gene expression levels;
   selecting rare variants from gene sequences of the outlier individuals, wherein the rare variants are selected based on an allele frequency cutoff;
   fitting a causality model to determine causal relationships between the rare variants and the extreme levels of gene expression in the outlier individuals while controlling for a plurality of confounders; and
   generating causality scores for the rare variants based on the determined causal relationships, wherein a particular causality score of a particular rare variant indicates a likelihood of the particular rare variant causing an extreme level of gene expression in those outlier individuals whose gene sequences contain the particular rare variant.
2. The computer-implemented method of clause 1, wherein the causality scores are probability values (p-values).
3. The computer-implemented method of clause 1, wherein the p-values are determined by a Pearson correlation coefficient.
4. The computer-implemented method of clause 1, wherein the causality model is a logistic regression model, a linear regression model, an analysis of covariance (ANCOVA) model, and/or a multivariate analysis of covariance (MANCOVA) model.
5. The computer-implemented method of clause 4, wherein the fitted causality model determines the causal relationships by predicting a particular gene expression level of a particular gene in a particular chromosome in dependence upon a variant-driven gene expression level caused by a particular rare variant.
6. The computer-implemented method of clause 5, wherein the fitted causality model measures a contribution of the variant-driven gene expression level as a variant effect covariate.
7. The computer-implemented method of clause 1, wherein the plurality of confounders includes distal trans-expression quantitative trait loci (eQTLs) effects.
8.The computer-implemented method of clause 7, wherein the fitted causality model controls for the distal trans-eQTLs effects by predicting the particular gene expression level in dependence upon a trans gene expression level caused by other genes in other chromosomes.
9. The computer-implemented method of clause 8, wherein the fitted causality model measures a contribution of the trans gene expression level as a trans effect covariate.
10. The computer-implemented method of clause 1, wherein the plurality of confounders includes local cis-eQTLs effects.
11. The computer-implemented method of clause 10, wherein the fitted causality model controls for the local cis-eQTLs effects by predicting the particular gene expression level in dependence upon a cis gene expression level caused by a presence of a plurality of common variants in a neighborhood of the particular gene.
12. The computer-implemented method of clause 11, wherein the neighborhood is defined by an offset from a transcription start site (TSS) in the particular gene.
13. The computer-implemented method of clause 11, wherein the fitted causality model measures a contribution of the cis gene expression level as a cis effect covariate.
14. The computer-implemented method of clause 1, wherein the plurality of confounders includes population structure and ancestry effects.
15. The computer-implemented method of clause 14, wherein the population structure and ancestry effects are represented by one or more genotype-based principal components (gPCs).
16. The computer-implemented method of clause 15, wherein the fitted causality model controls for the population structure and ancestry effects by predicting the particular gene expression level in dependence upon a gPC gene expression level caused by the gPCs.
17. The computer-implemented method of clause 16, wherein the fitted causality model measures a contribution of the gPC gene expression level as a population structure and ancestry effect covariate.
18. The computer-implemented method of clause 1, wherein the plurality of confounders includes probabilistic estimation of expression residuals (PEER) effects.
19. The computer-implemented method of clause 18, wherein the fitted causality model controls for the PEER effects by predicting the particular gene expression level in dependence upon a PEER gene expression level caused by the PEER.
20. The computer-implemented method of clause 19, wherein the fitted causality model measures a contribution of the PEER gene expression level as a PEER effect covariate.
21. The computer-implemented method of clause 1, wherein the plurality of confounders includes environmental effects.
22. The computer-implemented method of clause 21, wherein the fitted causality model controls for the environmental effects by predicting the particular gene expression level in dependence upon an environment gene expression level caused by the environmental effects.
23. The computer-implemented method of clause 22, wherein the fitted causality model measures a contribution of the environment gene expression level as an environmental effect covariate.
23. The computer-implemented method of clause 1, wherein the plurality of confounders includes gender effects, batch effects, genotyping platform effects, and library construction protocol effects.
24. The computer-implemented method of clause 1, wherein the extreme levels of gene expression include over gene expression and under gene expression.
25. The computer-implemented method of clause 24, further including determining the causal relationships between the rare variants and the over gene expression while controlling for the plurality of confounders.
26. The computer-implemented method of clause 25, further including generating over causality scores for the rare variants, wherein a particular over causality score of the particular rare variant indicates a likelihood of the particular rare variant causing an over gene expression in those outlier individuals whose gene sequences contain the particular rare variant.
27. The computer-implemented method of clause 26, wherein the over causality scores are over probability values (over p-values).
28. The computer-implemented method of clause 27, wherein the over p-values are determined by a Pearson correlation coefficient.
29. The computer-implemented method of clause 27, wherein the over p-values specify statistically unconfounded likelihoods of the rare variants increasing gene expression in genes that otherwise have lower gene expression relative to other genes in a gene set.
30. The computer-implemented method of clause 24, further including determining the causal relationships between the rare variants and the under gene expression while controlling for the plurality of confounders.
31. The computer-implemented method of clause 30, further including generating under causality scores for the rare variants, wherein a particular under causality score of the particular rare variant indicates a likelihood of the particular rare variant causing an under gene expression in those outlier individuals whose gene sequences contain the particular rare variant.
32. The computer-implemented method of clause 31, wherein the under causality scores are under probability values (under p-values).
33. The computer-implemented method of clause 32, wherein the under p-values are determined by a Pearson correlation coefficient.
34. The computer-implemented method of clause 32, wherein the under p-values specify statistically unconfounded likelihoods of the rare variants decreasing gene expression in genes that otherwise have higher gene expression relative to other genes in a gene set.
35. The computer-implemented method of clause 1, wherein the rare variants are non-coding variants.
36. The computer-implemented method of clause 17, wherein the non-coding variants include five prime untranslated region (UTR) variants, three prime UTR variants, enhancer variants, and promoter variants.
37. The computer-implemented method of clause 1, wherein the gene expression levels are further stratified into tissue-specific gene expression levels for a plurality of tissues.
38. The computer-implemented method of clause 37, wherein the gene expression levels for each gene in each tissue are normalized using quantile normalization.
39. The computer-implemented method of clause 38, wherein the causality model is fitted separately for each tissue.
40. The computer-implemented method of clause 1, wherein the causality model is fitted using stratification.
41. The computer-implemented method of clause 1, further including generating a ranking of the rare variants based on the causality scores.
42. The computer-implemented method of clause 26, further including generating a ranking of the rare variants based on the over causality scores.
43. The computer-implemented method of clause 31, further including generating a ranking of the rare variants based on the under causality scores.
44. The computer-implemented method of clause 1, wherein the rare variants are singleton variants.
45. The computer-implemented method of clause 44, wherein a singleton variant occurs in only one outlier individual from the outlier individuals.
46. A system including one or more processors coupled to memory, the memory loaded with computer instructions to identify rare variants that cause extreme levels of gene expression, the instructions, when executed on the processors, implement actions comprising:
   accessing gene expression levels for a group of individuals;
   normalizing the gene expression levels, and identifying those outlier individuals from the group of individuals that have extreme levels of gene expression, wherein the extreme levels of gene expression are determined from tail quantiles of the normalized gene expression levels;
   selecting rare variants from gene sequences of the outlier individuals, wherein the rare variants are selected based on an allele frequency cutoff;
   fitting a causality model to determine causal relationships between the rare variants and the extreme levels of gene expression in the outlier individuals while controlling for a plurality of confounders; and
   generating causality scores for the rare variants based on the determined causal relationships, wherein a particular causality score of a particular rare variant indicates a likelihood of the particular rare variant causing an extreme level of gene expression in those outlier individuals whose gene sequences contain the particular rare variant.
47. The system of clause 46, further implementing actions comprising executing clauses 2-45.
48. A non-transitory computer readable storage medium impressed with computer program instructions to identify rare variants that cause extreme levels of gene expression, the instructions, when executed on a processor, implement a method comprising:
   accessing gene expression levels for a group of individuals;
   normalizing the gene expression levels, and identifying those outlier individuals from the group of individuals that have extreme levels of gene expression, wherein the extreme levels of gene expression are determined from tail quantiles of the normalized gene expression levels;
   selecting rare variants from gene sequences of the outlier individuals, wherein the rare variants are selected based on an allele frequency cutoff;
   fitting a causality model to determine causal relationships between the rare variants and the extreme levels of gene expression in the outlier individuals while controlling for a plurality of confounders; and
   generating causality scores for the rare variants based on the determined causal relationships, wherein a particular causality score of a particular rare variant indicates a likelihood of the particular rare variant causing an extreme level of gene expression in those outlier individuals whose gene sequences contain the particular rare variant.
49. The non-transitory computer readable storage medium of clause 46, implementing the method further comprising executing clauses 2-45.

### Clauses Set 2

1. An artificial intelligence-based system to detect changes in gene expression at per-base resolution, comprising:
   an input generation logic that accesses a sequence database and generates an input base sequence, wherein the input base sequence includes a target base sequence, and wherein the target base sequence is flanked by a right base sequence with downstream context bases, and a left base sequence with upstream context bases;
   a chromatin model that processes the input base sequence and generates an alternative representation of the input base sequence; and
   a chromatin output generation logic that processes the alternative representation of the input base sequence and generates a output sequence of respective per-base chromatin outputs for respective target bases in the target base sequence,
      wherein a given per-base chromatin output in the output sequence for a given target base at a given position in the target base sequence specifies a measure of evolutionary conservation of the given target base across a plurality of species.
2. The artificial intelligence-based system of clause 1, wherein the given per-base chromatin output further specifies a measure of transcription initiation of the given target base at the given position.
3. The artificial intelligence-based system of clause 1, wherein the measure of evolutionary conservation is a phylogenetic P-values (phyloP) score that specifies a deviation from a null model of neural substitution to detect a reduction in a rate of substitution of the given target base at the given position as conservation, and to detect an increase in the rate of substitution of the given target base at the given position as acceleration.
4. The artificial intelligence-based system of clause 1, wherein the measure of evolutionary conservation is a phastCons score that specifies a posterior probability of the given target base at the given position having a conserved state or a non-conserved state.
5. The artificial intelligence-based system of clause 1, wherein the measure of evolutionary conservation is a genomic evolutionary rate profiling (GERP) score that specifies a reduction in a number of substitutions of the given target base at the given position across the plurality of species.
6. The artificial intelligence-based system of clause 2, wherein the measure of transcription initiation is a cap analysis of gene expression (CAGE) score that specifies a transcription initiation frequency of the given target base at the given position.
7. The artificial intelligence-based system of clause 1, wherein the given per-base chromatin output further specifies a confounder signal level for the given target base at the given position.
8. The artificial intelligence-based system of clause 7, wherein the confounder signal level specifies DNase I-hypersensitive sites (DHSs).
9. The artificial intelligence-based system of clause 7, wherein the confounder signal level specifies transcription factor (TF) bindings.
10. The artificial intelligence-based system of clause 7, wherein the confounder signal level specifies histone modification (HM) marks.
11. The artificial intelligence-based system of clause 7, wherein the confounder signal level specifies DNA methylation marks.
12. The artificial intelligence-based system of clause 1, further configured to comprise:
   a gene expression model that processes the output sequence and generates an alternative representation of the output sequence; and
   a gene expression model output generation logic that processes the alternative representation of the output sequence and generates a gene expression output sequence of respective per-base gene expression outputs for the respective target bases in the target base sequence,
   wherein a given per-base gene expression output in the gene expression output sequence for the given target base at the given position specifies a measure of gene expression level of the given target base at the given position.
13. The artificial intelligence-based system of clause 12, wherein the gene expression level is measured in a per-base metric such as CAGE transcription start site (CTSS).
14. The artificial intelligence-based system of clause 12, wherein the gene expression level is measured in a per-gene metric such as transcripts per million (TPM) or reads per kilobase of transcript (RPKM).
15. The artificial intelligence-based system of clause 12, wherein the gene expression level is measured in a per-gene metric such as fragments per kilobase million (FPKM).
16. The artificial intelligence-based system of clause 1, further configured to comprise a variant classification logic.
17. The artificial intelligence-based system of clause 16, wherein the variant classification logic is further configured to comprise a reference input generation logic that accesses the sequence database and generates a reference base sequence, wherein the reference base sequence includes a reference target base sequence, wherein the reference target base sequence includes a reference base at a position-under-analysis, and wherein the reference base is flanked by a right base sequence with downstream context bases, and a left base sequence with upstream context bases.
18. The artificial intelligence-based system of clause 17, wherein the variant classification logic is further configured to comprise an alternate input generation logic that accesses the sequence database and generates an alternate base sequence, wherein the alternate base sequence includes an alternate target base sequence, wherein the alternate target base sequence includes an alternate base at the position-under-analysis, and wherein the alternate base is flanked by the right base sequence with the downstream context bases, and the left base sequence with the upstream context bases.
19. The artificial intelligence-based system of clause 17, wherein the variant classification logic is further configured to comprise a reference processing logic that causes the chromatin model to process the reference base sequence and generate an alternative representation of the reference base sequence, and further causes the chromatin output generation logic to process the alternative representation of the reference base sequence and generate a reference output sequence of respective per-base reference chromatin outputs for respective reference target bases in the reference target base sequence,
   wherein a given per-base reference chromatin output in the reference output sequence for a given reference target base at a given position in the reference target base sequence specifies a measure of evolutionary conservation of the given reference target base across the plurality of species.
20. The artificial intelligence-based system of clause 18, wherein the variant classification logic is further configured to comprise an alternate processing logic that causes the chromatin model to process the alternate base sequence and generate an alternative representation of the alternate base sequence, and further causes the chromatin output generation logic to process the alternative representation of the alternate base sequence and generate an alternate output sequence of respective per-base alternate chromatin outputs for respective alternate target bases in the alternate target base sequence,
   wherein a given per-base alternate chromatin output in the alternate output sequence for a given alternate target base at a given position in the alternate target base sequence specifies a measure of evolutionary conservation of the given alternate target base across the plurality of species.
21. The artificial intelligence-based system of clause 20, wherein the variant classification logic is further configured to comprise a pathogenicity prediction logic that position-wise compares the reference output sequence and the alternate output sequence and generates a delta sequence with position-wise sequence diffs for positions in the reference output sequence and the alternate output sequence.
22. The artificial intelligence-based system of clause 21, wherein the pathogenicity prediction logic is further configured to generate a pathogenicity prediction for the alternate base in dependence upon the delta sequence.
23. The artificial intelligence-based system of clause 21, wherein the pathogenicity prediction logic is further configured to accumulate the position-wise sequence diffs into an accumulated sequence value and generate the pathogenicity prediction for the alternate base in dependence upon the accumulated sequence value.
24. The artificial intelligence-based system of clause 23, wherein the accumulated sequence value is an average or max of the position-wise sequence diffs.
25. The artificial intelligence-based system of clause 23, wherein the accumulated sequence value is a sum of the position-wise sequence diffs.
26. The artificial intelligence-based system of clause 21, wherein the pathogenicity prediction logic is further configured to position-wise compare respective portions of the reference output sequence and the alternate output sequence and generate a delta sub-sequence with position-wise sub-sequence diffs for positions in the respective portions.
27. The artificial intelligence-based system of clause 26, wherein the respective portions span right and left flanking positions around the position-under-analysis.
28. The artificial intelligence-based system of clause 26, wherein the pathogenicity prediction logic is further configured to generate a pathogenicity prediction for the alternate base in dependence upon the delta sub-sequence.
29. The artificial intelligence-based system of clause 26, wherein the pathogenicity prediction logic is further configured to accumulate the position-wise sub-sequence diffs into an accumulated sub-sequence value and generate the pathogenicity prediction for the alternate base in dependence upon the accumulated sub-sequence value.
30. The artificial intelligence-based system of clause 29, wherein the accumulated sub-sequence value is an average of the position-wise sub-sequence diffs.
31. The artificial intelligence-based system of clause 29, wherein the accumulated sub-sequence value is a sum or max of the position-wise sub-sequence diffs.
32. The artificial intelligence-based system of clause 1, wherein the target base sequence is a coding region of a gene.
33. The artificial intelligence-based system of clause 1, wherein the target base sequence is a non-coding region of a gene.
34. The artificial intelligence-based system of clause 33, wherein the non-coding region spans transcription start sites, five prime untranslated region (UTRs), three prime UTRs, enhancers, and promoters.
35. The artificial intelligence-based system of clause 18, wherein the alternate base is a rare variant an outlier individual among a cohort of outlier individuals,
   wherein outlier individuals in the cohort of outlier individuals exhibit extremes levels of gene expression.
36. The artificial intelligence-based system of clause 35, wherein the extremes levels of gene expression are determined from tail quantiles of normalized gene expression levels.
37. The artificial intelligence-based system of clause 36, wherein the extremes levels of gene expression include over gene expression and under gene expression.
38. The artificial intelligence-based system of clause 35, wherein the rare variant is a coding variant.
39. The artificial intelligence-based system of clause 35, wherein the rare variant is a non-coding variant.
40. The artificial intelligence-based system of clause 39, wherein the non-coding variant is a promoter variant.
41. The artificial intelligence-based system of clause 39, wherein the non-coding variant is an enhancer variant.
42. The artificial intelligence-based system of clause 1, wherein the chromatin model has a first set of weights,
   wherein the chromatin output generation logic has a second set of weights.
43. The artificial intelligence-based system of clause 42, wherein, during training, the first set of weights of the chromatin model is trained from scratch to process the input base sequence and generate the alternative representation of the input base sequence,
   wherein the second set of weights of the chromatin output generation logic is trained from scratch and end-to-end with the first set of weights of the chromatin model to process the alternative representation of the input base sequence and generate the output sequence.
44. The artificial intelligence-based system of clause 63, wherein, during inference, the chromatin model uses the trained first set of weights,
   wherein, during the inference, the chromatin output generation logic uses the trained second set of weights.
45. The artificial intelligence-based system of clause 1, wherein the gene expression model has a third set of weights,
   wherein the gene expression output generation logic has a fourth set of weights.
46. The artificial intelligence-based system of clause 45, wherein the third set of weights of the gene expression model is trained from scratch to process the output sequence and generate the alternative representation of the output sequence,
   wherein the fourth set of weights of the gene expression output generation logic is trained from scratch and end-to-end with the third set of weights of the gene expression model to process the alternative representation of the output sequence and generate the gene expression output sequence.
47. The artificial intelligence-based system of clause 46, wherein, during inference, the gene expression model uses the trained third set of weights,
   wherein, during the inference, the gene expression output generation logic uses the trained fourth set of weights.
48. The artificial intelligence-based system of clause 45, wherein, during training, the first set of weights of the chromatin model is first trained to process the input base sequence and generate the alternative representation of the input base sequence, and then retrained as a substitute of the third set of weights of the gene expression model to process the output sequence and generate the alternative representation of the output sequence,
   wherein the fourth set of weights of the gene expression output generation logic is trained from scratch and end-to-end with the trained first set of weights substituted in the gene expression model to process the alternative representation of the output sequence generated by the trained first set of weights substituted in the gene expression model and generate the gene expression output sequence.
49. The artificial intelligence-based system of clause 48, wherein, during inference, the chromatin model uses the retrained first set of weights,
   wherein, during the inference, the chromatin output generation logic uses the trained second set of weights,
   wherein, during inference, the gene expression model uses the retrained first set of weights, and
   wherein, during the inference, the gene expression output generation logic uses the trained fourth set of weights.
50. The artificial intelligence-based system of clause 19, wherein, during training, the first set of weights of the chromatin model is first trained from scratch to process the input base sequence and generate the alternative representation of the input base sequence,
   wherein, during the training, the second set of weights of the chromatin output generation logic is first trained from scratch and end-to-end with the first set of weights of the chromatin model to process the alternative representation of the input base sequence and generate the output sequence,
   wherein, during the training, the trained first set of weights of the chromatin model is then retrained to process the reference base sequence and generate the alternative representation of the reference base sequence, and to process the alternate base sequence and generate the alternative representation of the alternate base sequence, and
   wherein, during the training, the trained second set of weights of the chromatin output generation logic is then retrained end-to-end with the trained first set of weights of the chromatin model to process the alternative representation of the reference base sequence and generate the reference output sequence, and to process the alternative representation of the alternate base sequence and generate the alternate output sequence.
51. The artificial intelligence-based system of clause 50, wherein, during inference, the chromatin model uses the retrained first set of weights,
   wherein, during the inference, the chromatin output generation logic uses the retrained second set of weights.
52. The artificial intelligence-based system of clause 21, wherein the pathogenicity prediction logic has a fifth set of weights.
53. The artificial intelligence-based system of clause 52, wherein, during training, the first set of weights of the chromatin model is first trained from scratch to process the input base sequence and generate the alternative representation of the input base sequence,
   wherein, during the training, the second set of weights of the chromatin output generation logic is first trained from scratch and end-to-end with the first set of weights of the chromatin model to process the alternative representation of the input base sequence and generate the output sequence, and
   wherein, during the training, the trained first set of weights of the chromatin model and the trained second set of weights of the chromatin output generation logic are then retrained end-to-end to generate the pathogenicity prediction for the alternate base.
54. The artificial intelligence-based system of clause 53, wherein, during inference, the chromatin model uses the retrained first set of weights,
   wherein, during the inference, the chromatin output generation logic uses the retrained second set of weights, and
   wherein, during the inference, the pathogenicity prediction logic uses the trained fifth set of weights.
55. The artificial intelligence-based system of clause 19, wherein the given per-base reference chromatin output in the reference output sequence for the given reference target base at the given position in the reference target base sequence specifies a confounder signal level for the given reference target base at the given position.
56. The artificial intelligence-based system of clause 20, wherein the given per-base alternate chromatin output in the alternate output sequence for the given alternate target base at the given position in the alternate target base sequence specifies a confounder signal level for the given alternate target base at the given position.
57. The artificial intelligence-based system of clause 1, wherein, during training, the chromatin model and the chromatin output generation logic are first trained from scratch and end-to-end to translate analysis of input base sequences into base-wise evolutionary conservation chromatin sequences, and then retrained end-to-end to translate analysis of input base sequences into base-wise transcription initiation frequency chromatin sequences.
58. The artificial intelligence-based system of clause 1, wherein, during training, the chromatin model and the chromatin output generation logic are first trained from scratch and end-to-end to translate analysis of input base sequences into base-wise confounder signal level chromatin sequences, and then retrained end-to-end to translate analysis of input base sequences into base-wise evolutionary conservation chromatin sequences.
59. The artificial intelligence-based system of clause 1, wherein, during training, the chromatin model and the chromatin output generation logic are first trained from scratch and end-to-end to translate analysis of input base sequences into base-wise confounder signal level chromatin sequences, and then retrained end-to-end to translate analysis of input base sequences into base-wise transcription initiation frequency chromatin sequences.
60. The artificial intelligence-based system of clause 1, wherein, during training, the chromatin model and the chromatin output generation logic are first trained from scratch and end-to-end to translate analysis of input base sequences into base-wise confounder signal level chromatin sequences, and then retrained end-to-end to translate analysis of input base sequences into base-wise evolutionary conservation chromatin sequences and base-wise transcription initiation frequency chromatin sequences.
61. The artificial intelligence-based system of clause 1, further configured to comprise a first training set of training input base sequences that include variants confounded by a plurality of confounder effects.
62. The artificial intelligence-based system of clause 61, wherein confounder effects in the plurality of confounder effects include inter-chromosomal effects, intra-gene effects, population structure and ancestry effects, probabilistic estimation of expression residuals (PEER) effects, environmental effects, gender effects, batch effects, genotyping platform effects, and/or library construction protocol effects.
63. The artificial intelligence-based system of clause 61, further configured to comprise a second training set of training input base sequences that include variants unconfounded by the plurality of confounder effects.
64. The artificial intelligence-based system of clause 63, wherein the variants in the second training set are reliably determined to alter gene expression and cause extreme levels of gene expression.
65. The artificial intelligence-based system of clause 64, wherein the variants in the second training set include over expression-causing variants that increase gene expression levels.
66. The artificial intelligence-based system of clause 64, wherein the variants in the second training set include under expression-causing variants that decrease gene expression levels.
67. The artificial intelligence-based system of clause 65, wherein the second training set specifies over expression probabilities for the variants that specify likelihoods of the causing gene over expression.
68. The artificial intelligence-based system of clause 66, wherein the second training set specifies under expression probabilities for the variants that specify likelihoods of the variants causing gene under expression.
69. The artificial intelligence-based system of clause 64, wherein each variant in the second training set is a rare variant that occurs in an outlier individual among a cohort of outlier individuals,
   wherein outlier individuals in the cohort of outlier individuals exhibit extremes levels of gene expression.
70. The artificial intelligence-based system of clause 69, wherein the extremes levels of gene expression are determined from tail quantiles of normalized gene expression levels.
71. The artificial intelligence-based system of clause 70, wherein the extremes levels of gene expression include over gene expression and under gene expression.
72. The artificial intelligence-based system of clause 69, wherein the rare variant is a coding variant.
73. The artificial intelligence-based system of clause 69, wherein the rare variant is a non-coding variant.
74. The artificial intelligence-based system of clause 73, wherein the non-coding variant is a five prime untranslated region (UTR) variant, a three prime UTR variant, an enhancer variant, or a promoter variant.
75. The artificial intelligence-based system of clause 64, wherein the variants in the second training set span a plurality of tissue types.
76. The artificial intelligence-based system of clause 64, wherein the variants in the second training set span a plurality of cell types.
77. The artificial intelligence-based system of clause 1, wherein the input base sequences and the plurality of output sequences span the plurality of tissue types.
78. The artificial intelligence-based system of clause 1, wherein the input base sequences and the plurality of output sequences span the plurality of cell types.
79. The artificial intelligence-based system of clause 12, wherein the gene expression output sequence spans the plurality of tissue types.
80. The artificial intelligence-based system of clause 12, wherein the gene expression output sequence spans the plurality of cell types.
81. The artificial intelligence-based system of clause 1, wherein the chromatin model and the chromatin output generation logic are first trained end-to-end on the first training set, and then retrained on the second training set.
82. The artificial intelligence-based system of clause 1, wherein the variants in the second training set are used as a pathogenic set labelled with a first ground truth label indicating gene expression alteration, and common variants are used as a benign set labelled with a second ground truth label indicating gene expression non-alteration.
83. The artificial intelligence-based system of clause 102, wherein the benign set is balanced for trinucleotide context, homopolymers, k-mers, neighborhood GC frequency, and sequencing depth.
84. The artificial intelligence-based system of clause 102, wherein, based on a cutoff probability applied to the over expression probabilities and the under expression probabilities, the variants in the second training set are partitioned into an over expression variant training set with a first ground truth label indicating gene expression increase, an over expression variant training set with a second ground truth label indicating gene expression reduction, and a neural expression variant training set indicating gene expression maintenance.
85. The artificial intelligence-based system of clause 12, wherein the gene expression model and the gene expression output generation logic are first trained end-to-end on the first training set, and then retrained on the second training set.
86. The artificial intelligence-based system of clause 1, wherein the chromatin model and the chromatin output generation logic are first trained end-to-end on the first training set, and then retrained on those variants in the second training set that occur on odd chromosomes.
87. The artificial intelligence-based system of clause 12, wherein the gene expression model and the gene expression output generation logic are first trained end-to-end on the first training set, and then retrained on those variants in the second training set that occur on odd chromosomes.
88. The artificial intelligence-based system of clause 64, wherein the variants in the second training set are not used for training instead used as a validation set to evaluate performance of the trained chromatin model, the trained the chromatin output generation logic, the trained gene expression model, and the trained gene expression output generation logic.
89. The artificial intelligence-based system of clause 88, wherein those variants in the second training set that occur on even chromosomes are used as the validation set.
90. The artificial intelligence-based system of clause 1, wherein a size of the target base sequence varies during training to account for varying offset locations of transcription start sites (TSSs).
91. An artificial intelligence-based system to detect changes in gene expression at per-base resolution, comprising:
   an input generation logic that accesses a sequence database and generates an input base sequence, wherein the input base sequence includes a target base sequence, and wherein the target base sequence is flanked by a right base sequence with downstream context bases, and a left base sequence with upstream context bases;
   a chromatin model that processes the input base sequence and generates an alternative representation of the input base sequence; and
   an output generation logic that processes the alternative representation of the input base sequence and generates a output sequence of respective per-base chromatin outputs for respective target bases in the target base sequence,
      wherein a given per-base chromatin output in the output sequence for a given target base at a given position in the target base sequence specifies a measure of transcription initiation of the given target base at the given position.
92. An artificial intelligence-based system to detect changes in gene expression at per-base resolution, comprising:
   an input generation logic that accesses a sequence database and generates an input base sequence, wherein the input base sequence includes a target base sequence, and wherein the target base sequence is flanked by a right base sequence with downstream context bases, and a left base sequence with upstream context bases;
   a gene expression model that processes the input base sequence and generates an alternative representation of the input base sequence; and
   a gene expression model output generation logic that processes the alternative representation of the input base sequence and generates a gene expression output sequence of respective per-base gene expression outputs for respective target bases in the target base sequence,
      wherein a given per-base gene expression output in the gene expression output sequence for the given target base at the given position specifies a measure of gene expression level of the given target base at the given position.
93. An artificial intelligence-based system to detect changes in gene expression at per-base resolution, comprising:
   an input generation logic that accesses a sequence database and generates an input base sequence, wherein the input base sequence includes a target base sequence, and wherein the target base sequence is flanked by a right base sequence with downstream context bases, and a left base sequence with upstream context bases;
   a chromatin model that processes the input base sequence and generates an alternative representation of the input base sequence; and
   a chromatin output generation logic that processes the alternative representation of the input base sequence and generates a output sequence of respective per-base chromatin outputs for respective target bases in the target base sequence.
94. The artificial intelligence-based system of clause 93, wherein a given per-base chromatin output in the output sequence for a given target base at a given position in the target base sequence specifies a measure of evolutionary conservation of the given target base across a plurality of species.
95. The artificial intelligence-based system of clause 93, wherein the given per-base chromatin output further specifies a measure of transcription initiation of the given target base at the given position.

While the present invention is disclosed by reference to the preferred implementations and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims.

1. A computer-implemented method of identifying rare variants that cause extreme levels of gene expression, including:
accessing gene expression levels for a group of individuals;
normalizing the gene expression levels, and identifying those outlier individuals from the group of individuals that have extreme levels of gene expression, wherein the extreme levels of gene expression are determined from tail quantiles of the normalized gene expression levels;
selecting rare variants from gene sequences of the outlier individuals, wherein the rare variants are selected based on an allele frequency cutoff;
fitting a causality model to determine causal relationships between the rare variants and the extreme levels of gene expression in the outlier individuals while controlling for a plurality of confounders; and
generating causality scores for the rare variants based on the determined causal relationships, wherein a particular causality score of a particular rare variant indicates a likelihood of the particular rare variant causing an extreme level of gene expression in those outlier individuals whose gene sequences contain the particular rare variant.

2. The computer-implemented method of claim 1, wherein the causality scores are probability values (p-values).

3. The computer-implemented method of claim 1, wherein the p-values are determined by a Pearson correlation coefficient.

4. The computer-implemented method of claim 1, wherein the causality model is a logistic regression model, a linear regression model, an analysis of covariance (ANCOVA) model, and/or a multivariate analysis of covariance (MANCOVA) model.

5. The computer-implemented method of claim 4, wherein the fitted causality model determines the causal relationships by predicting a particular gene expression level of a particular gene in a particular chromosome in dependence upon a variant-driven gene expression level caused by a particular rare variant.

6. (canceled).

7. The computer-implemented method of claim 1, wherein the plurality of confounders includes distal trans-expression quantitative trait loci (eQTLs) effects.

8. The computer-implemented method of claim 7, wherein the fitted causality model controls for the distal trans-eQTLs effects by predicting the particular gene expression level in dependence upon a trans gene expression level caused by other genes in other chromosomes.

9. (canceled)

10. The computer-implemented method of claim 1, wherein the plurality of confounders includes local cis-eQTLs effects.

11-13. (canceled)

14. The computer-implemented method of claim 1, wherein the plurality of confounders includes population structure and ancestry effects.

15-17. (canceled)

18. The computer-implemented method of claim 1, wherein the plurality of confounders includes probabilistic estimation of expression residuals (PEER) effects.

19-20. (canceled)

21. The computer-implemented method of claim 1, wherein the plurality of confounders includes environmental effects.

22-23. (canceled)

24. The computer-implemented method of claim 1, wherein the plurality of confounders includes gender effects, batch effects, genotyping platform effects, and library construction protocol effects.

25. The computer-implemented method of claim 1, wherein the extreme levels of gene expression include over gene expression and under gene expression.

26-35. (canceled)

36. The computer-implemented method of claim 1, wherein the rare variants are non-coding variants.

37. (canceled)

38. The computer-implemented method of claim 1, wherein the gene expression levels are further stratified into tissue-specific gene expression levels for a plurality of tissues.

39-40. (canceled)

41. The computer-implemented method of claim 1, wherein the causality model is fitted using stratification.

42. The computer-implemented method of claim 1, further including generating a ranking of the rare variants based on the causality scores.

43-44. (canceled)

45. The computer-implemented method of claim 1, wherein the rare variants are singleton variants.

46. (canceled)

47. A system including one or more processors coupled to memory, the memory loaded with computer instructions to identify rare variants that cause extreme levels of gene expression, the instructions, when executed on the processors, implement actions comprising:
accessing gene expression levels for a group of individuals;
normalizing the gene expression levels, and identifying those outlier individuals from the group of individuals that have extreme levels of gene expression, wherein the extreme levels of gene expression are determined from tail quantiles of the normalized gene expression levels;
selecting rare variants from gene sequences of the outlier individuals, wherein the rare variants are selected based on an allele frequency cutoff;
fitting a causality model to determine causal relationships between the rare variants and the extreme levels of gene expression in the outlier individuals while controlling for a plurality of confounders; and
generating causality scores for the rare variants based on the determined causal relationships, wherein a particular causality score of a particular rare variant indicates a likelihood of the particular rare variant causing an extreme level of gene expression in those outlier individuals whose gene sequences contain the particular rare variant.

48. (canceled)

49. A non-transitory computer readable storage medium impressed with computer program instructions to identify rare variants that cause extreme levels of gene expression, the instructions, when executed on a processor, implement a method comprising:
accessing gene expression levels for a group of individuals;
normalizing the gene expression levels, and identifying those outlier individuals from the group of individuals that have extreme levels of gene expression, wherein the extreme levels of gene expression are determined from tail quantiles of the normalized gene expression levels;
selecting rare variants from gene sequences of the outlier individuals, wherein the rare variants are selected based on an allele frequency cutoff;
fitting a causality model to determine causal relationships between the rare variants and the extreme levels of gene expression in the outlier individuals while controlling for a plurality of confounders; and
generating causality scores for the rare variants based on the determined causal relationships, wherein a particular causality score of a particular rare variant indicates a likelihood of the particular rare variant causing an extreme level of gene expression in those outlier individuals whose gene sequences contain the particular rare variant.

50. (canceled)
